(19) United States
(12) Patent Application Publication
(10) Pub. No.: US 2007/0093805 A1
(43) Pub. Date: Apr. 26, 2007

(54) Systems and methods for securing cardiovascular tissue, including via asymmetric electrodes

(75) Inventors: David C. Auth, Kirkland, WA (US); Christopher C. Genau, Seattle, WA (US); Joseph E. Eichinger, Everett, WA (US); Mark A. Tempel, Sammamish, WA (US); Ryan E. Kaveckis, Everett, WA (US); William Gray, New York, NY (US); Blair Erbstoeszer JR., Kirkland, WA (US)

Correspondence Address: PERKINS COIE LLP;PATENT-SEA, P.O. BOX 1247, SEATTLE, WA, 98111-1247, US

(73) Assignee: CoAptus Medical Corporation, Redmond, WA (US)

(21) Appl. No.: 11/582,210

(22) Filed: Oct. 16, 2006

Related U.S. Application Data
(60) Provisional application No. 60/727,678, filed on Oct. 17, 2005.
(60) Provisional application No. 60/811,866, filed on Jun. 7, 2006.
(60) Provisional application No. 60/811,993, filed on Jun. 7, 2006.
(60) Provisional application No. 60/811,864, filed on Jun. 7, 2006.
(60) Provisional application No. 60/811,999, filed on Jun. 7, 2006.
(60) Provisional application No. 60/812,002, filed on Jun. 7, 2006.

(51) Int. Cl. A61B 18/14

(52) U.S. Cl. 606/41; 606/49

(57) ABSTRACT

Systems and methods for securing cardiovascular tissue, including via asymmetric electrodes, are disclosed. A device in accordance with one embodiment includes a catheter having a proximal end and a distal end, with a working portion positioned toward the distal end and being elongated along a terminal axis. The device can further include an energy transmitter (e.g., an electrode) at the working portion of the catheter, with the energy transmitter tapered inwardly toward the terminal axis in a distal direction. The energy transmitter can be asymmetric relative to the terminal axis. In further particular embodiments, other components of the catheter (e.g., an inflatable member, guidewire conduit, and/or catheter bend angle) can also be asymmetric relative to the terminal axis, and in still further particular embodiments, some or all of the foregoing elements can have particular alignments relative to each other.

### CROSS-REFERENCE TO RELATED APPLICATIONS

The present application claims priority to U.S. Provisional Application 60/727,678 (filed on Oct. 17, 2005); and the following U.S. Provisional Applications, all filed on Jun. 7, 2006: 60/811,866; 60/811,993; 60/811,864; 60/811,999; and 60/812,002. All the foregoing applications are incorporated herein by reference.

### TECHNICAL FIELD

The present disclosure is directed generally to systems and methods for securing cardiovascular tissue, including via asymmetric electrodes.

### BACKGROUND

The human heart is a complex organ that requires reliable, fluid-tight seals to prevent de-oxygenated blood and other constituents received from the body's tissues from mixing with re-oxygenated blood delivered to the body's tissues. FIG. 1A illustrates a human heart 100 having a right atrium 101, which receives the de-oxygenated blood from the superior vena cava 116 and the inferior vena cava 104. The de-oxygenated blood passes to the right ventricle 103, which pumps the de-oxygenated blood to the lungs via the pulmonary artery 114. Re-oxygenated blood returns from the lungs to the left atrium 102 and is pumped into the left ventricle 105. From the left ventricle 105, the re-oxygenated blood is pumped throughout the body via the aorta 115.

The right atrium 101 and the left atrium 102 are separated by an interatrial septum 106. As shown in FIG. 1B, the interatrial septum 106 includes a primum 107 and a secundum 108. Prior to birth, the primum 107 and the secundum 108 are separated to form an opening (the foramen ovale 109) that allows blood to flow from the right atrium 101 to the left atrium 102 while the fetus receives oxygenated blood from the mother. After birth, the primum 107 normally seals against the secundum 108 and forms an oval-shaped depression, i.e., a fossa ovalis 110.

In some infants, the primum 107 never completely seals with the secundum 108, as shown in cross-sectional view in FIG. 1C and in a left side view in FIG. 1D. In these instances, a patency 111 often having the shape of a tunnel 112 forms between the primum 107 and the secundum 108. This patency is typically referred to as a patent foramen ovale or PFO 113. In most circumstances, the PFO 113 will remain functionally closed and blood will not tend to flow through the PFO 113, due to the higher pressures in the left atrium 102 that secure the primum 107 against the secundum 108. Nevertheless, during physical exertion or other instances when pressures are greater in the right atrium 101 than in the left atrium 102, blood can inappropriately pass directly from the right atrium 101 to the left atrium 102 and can carry with it clots, gas bubbles, or other vaso-active substances. Such constituents in the atrial system can pose serious health risks including hemodynamic problems, cryptogenic strokes, venous-to-atrial gas embolisms, migraines, and in some cases even death.

Traditionally, open chest surgery was required to suture or ligate a PFO 113. However, these procedures carry high attendant risks, such as postoperative infection, long patient recovery, and significant patient discomfort and trauma. Accordingly, less invasive techniques have been developed. Most such techniques include using transcatheter implantation of various mechanical devices to close the PFO 113. Such devices include the Cardia® PFO Closure Device, Amplatzer® PFO Occluder, and CardioSEAL® Septal Occlusion Device. One potential drawback with these devices is that they may not be well suited for the long, tunnel-like shape of the PFO 113. As a result, the implanted mechanical devices may become deformed or distorted and in some cases may fail, migrate, or even dislodge. Furthermore, these devices can irritate the cardiac tissue at or near the implantation site, which in turn can potentially cause thromboembolic events, palpitations, and arrhythmias. Other reported complications include weakening, erosion, and tearing of the cardiac tissues around the implanted devices.

Another potential drawback with the implanted mechanical devices described above is that, in order to be completely effective, the tissue around the devices must endothelize once the devices are implanted. The endothelization process can be gradual and can accordingly take several months or more to occur. Accordingly, the foregoing techniques do not immediately solve the problems caused by the PFO 113.

Still another drawback associated with the foregoing techniques is that they can be technically complicated and cumbersome. Accordingly, the techniques may require multiple attempts before the mechanical device is appropriately positioned and implanted. As a result, implanting these devices may require long procedure times during which the patient must be kept under conscious sedation, which can pose further risks to the patient.

### BRIEF DESCRIPTION OF THE DRAWINGS

FIGS. 1A-1D illustrate a human heart having a patent foramen ovale (PFO) in accordance with the prior art.

FIG. 2 illustrates a catheter configured in accordance with an embodiment of the invention and positioned proximate to a PFO.

FIG. 3 is an isometric illustration of a working portion of the catheter shown in FIG. 2.

FIG. 4 is a partial cross-sectional side elevation view of the working portion shown in FIG. 3.

FIGS. 5A and 5B illustrate the operation of a catheter in accordance with an embodiment of the invention.

FIG. 6A is an end view of a catheter working portion configured in accordance with further embodiments of the invention.

FIGS. 6B-6C illustrate an electrode coupled to a deployable catheter in accordance with another embodiment of the invention.

FIG. 6D illustrates a front isometric view of a catheter having an inflatable member tilted in accordance with another embodiment of the invention.

FIG. 6E illustrates a catheter having an inflatable member shaped in accordance with another embodiment of the invention.

FIG. 6F is a side view of a catheter having an electrode with a concave upper surface in accordance with another embodiment of the invention.

FIG. 6G is a rear isometric illustration of a catheter working portion carrying an inflatable member having ribs in accordance with another embodiment of the invention.

FIG. 6H is a cross-sectional, isometric illustration of an inflatable member having portions with different wall thicknesses in accordance with another embodiment of the invention.

FIG. 61 is a cross-sectional, isometric illustration of a working portion having an inflatable member with multiple chambers in accordance with another embodiment of the invention.

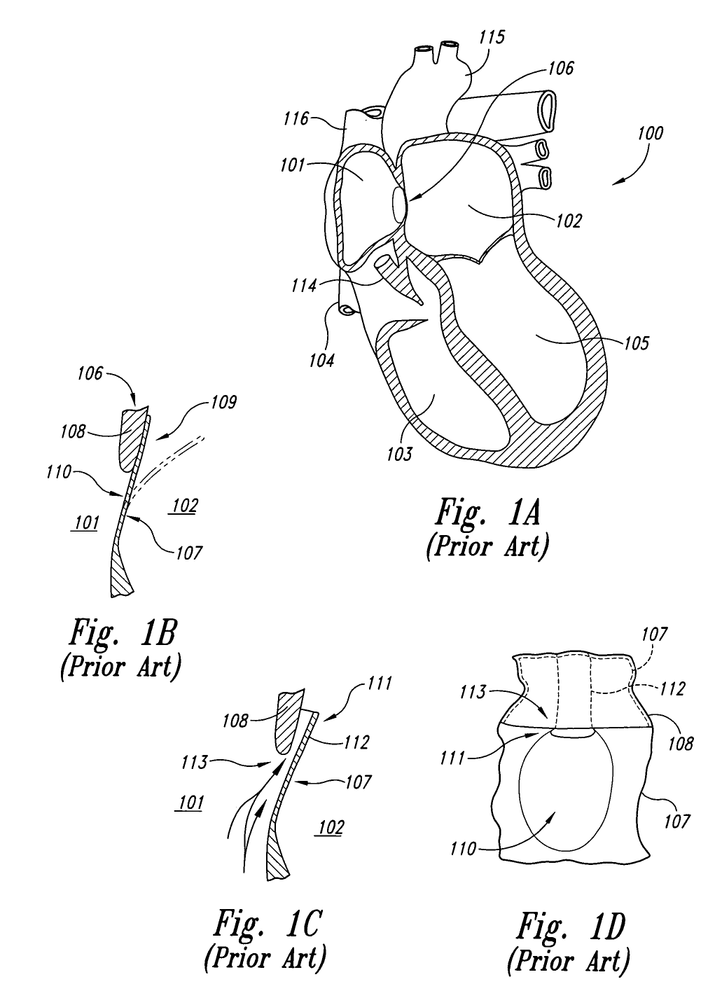

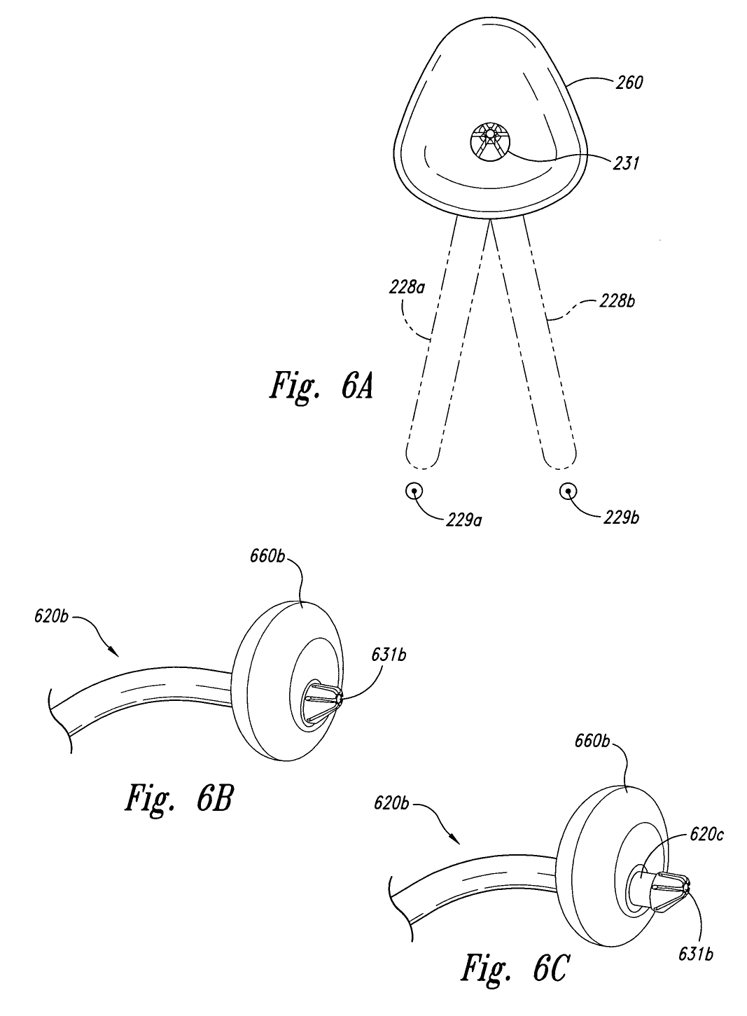

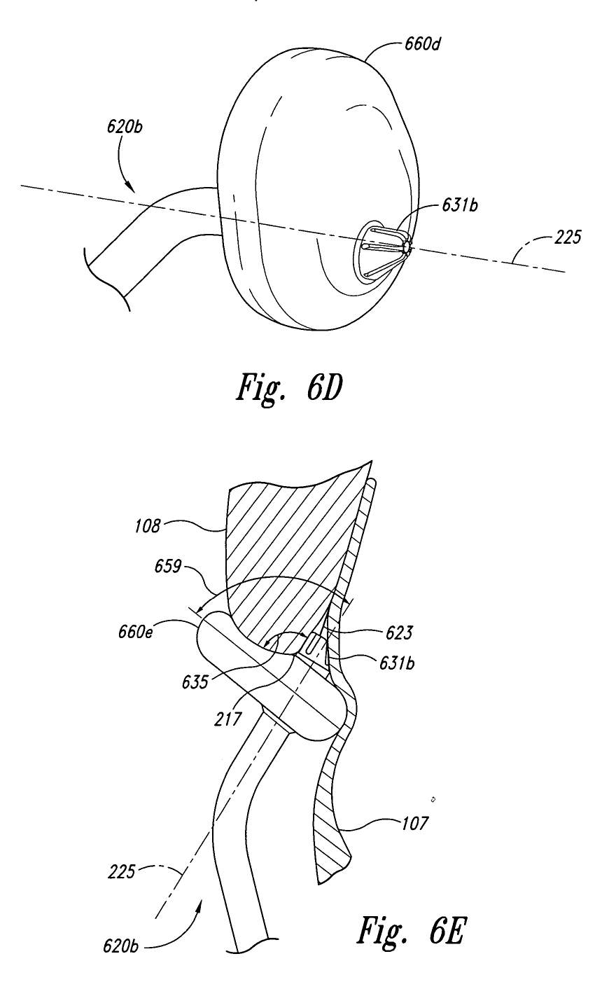

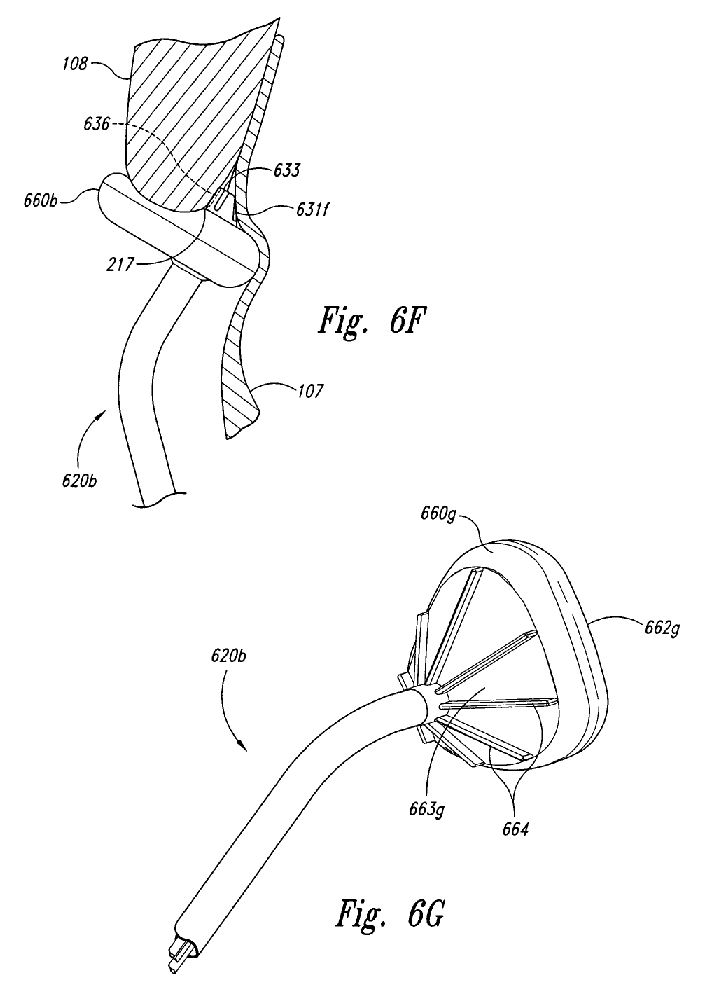

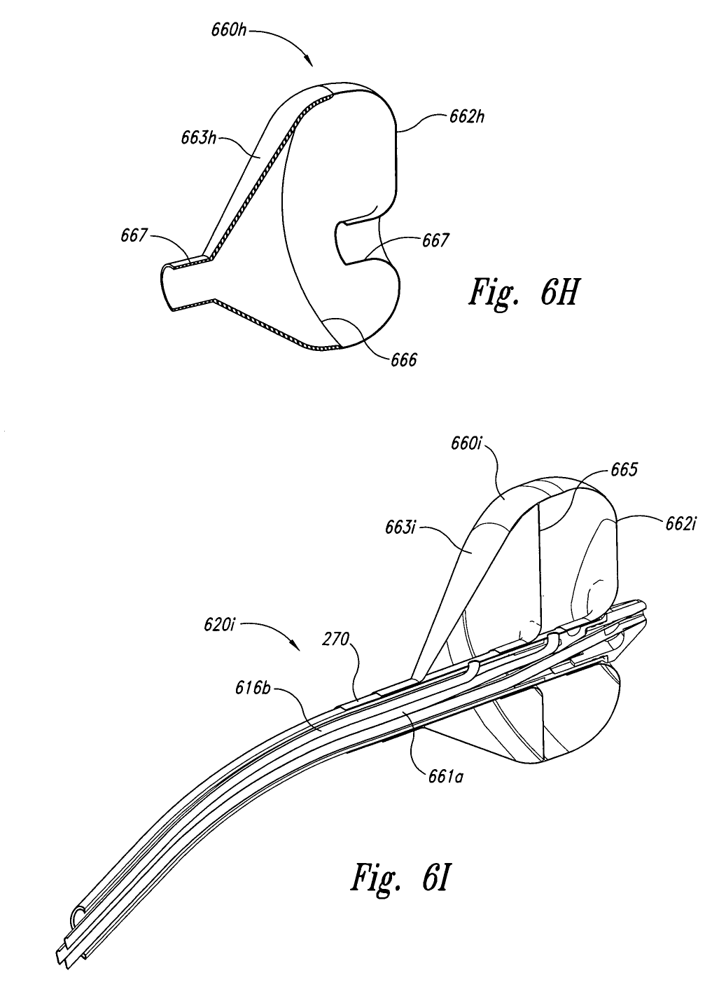

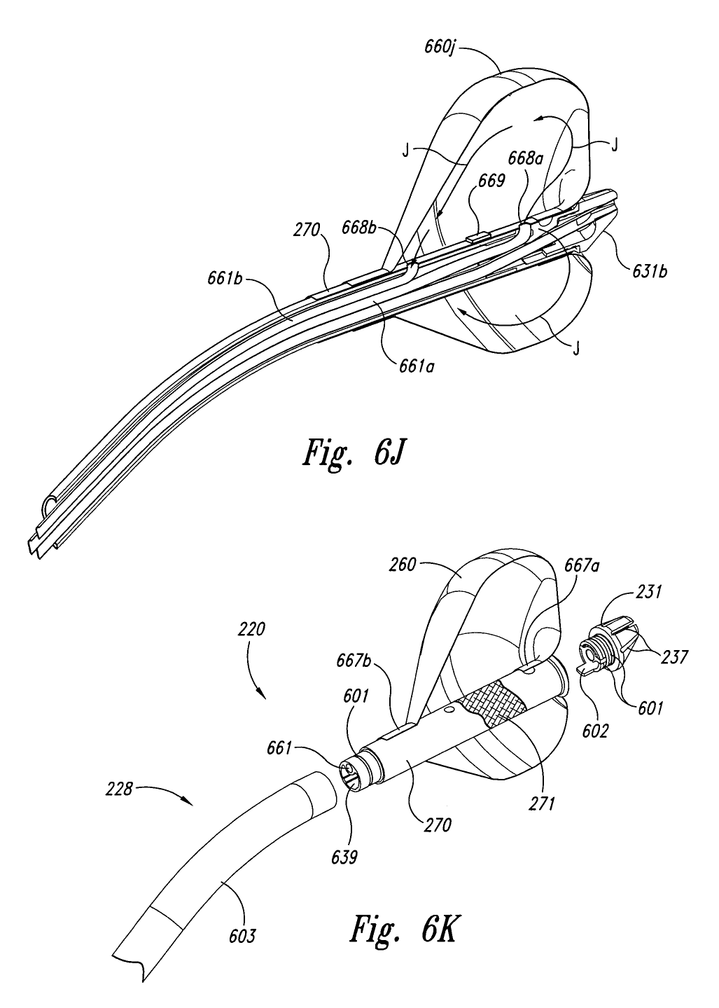

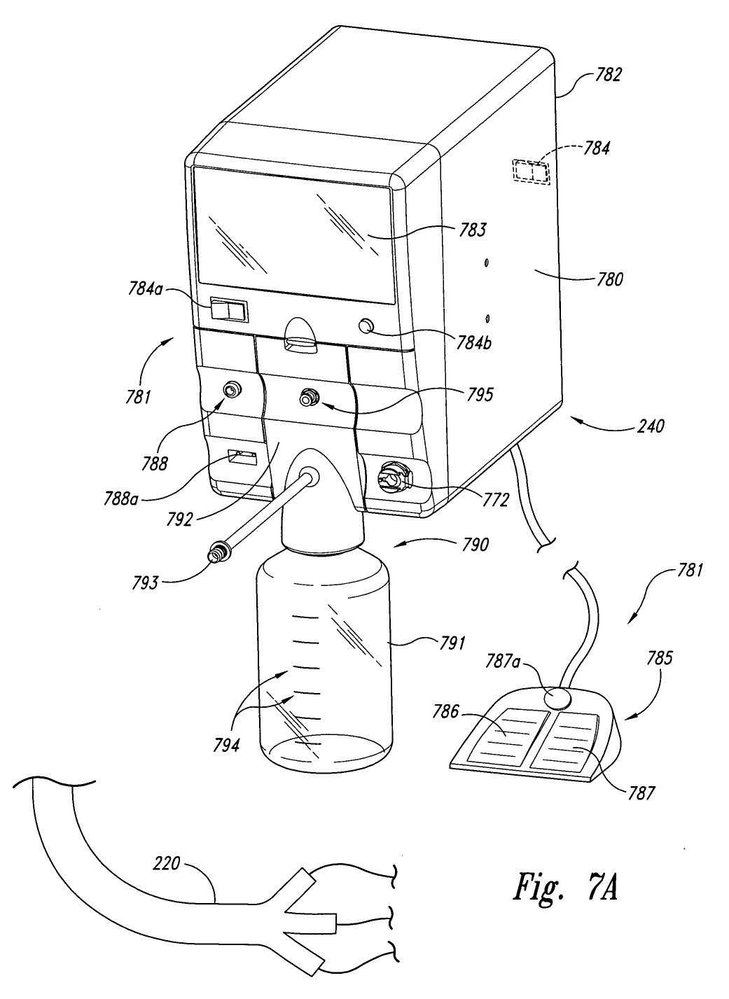

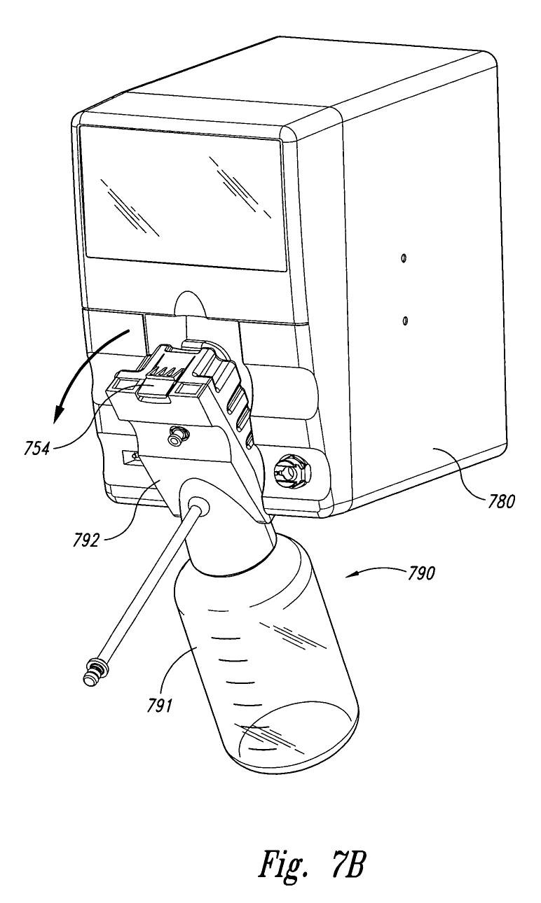

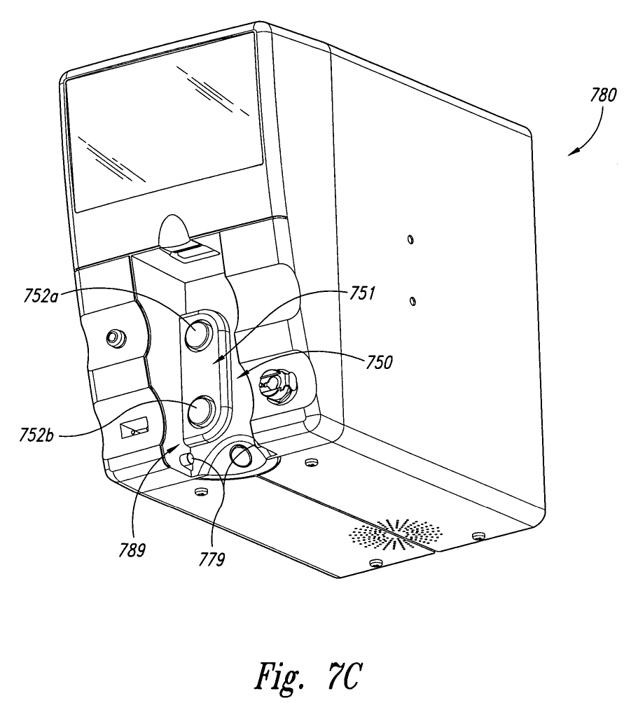

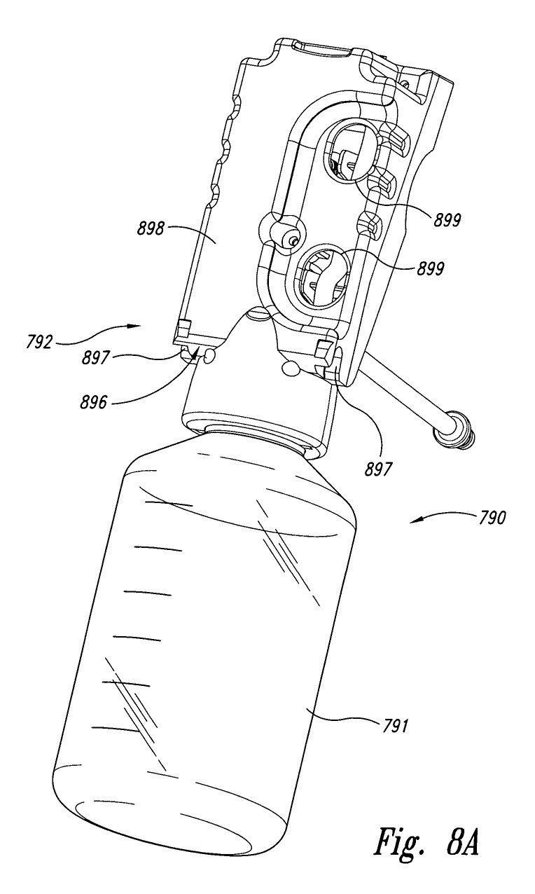

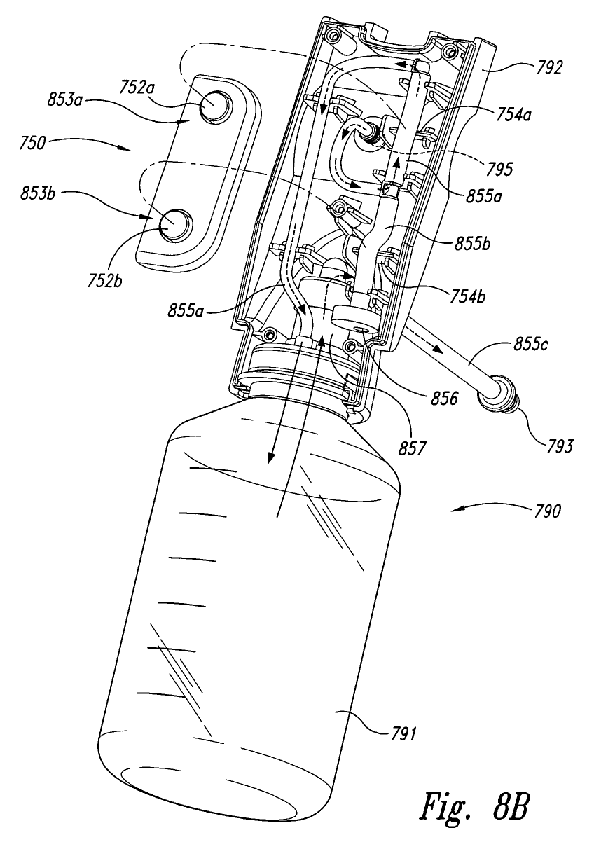

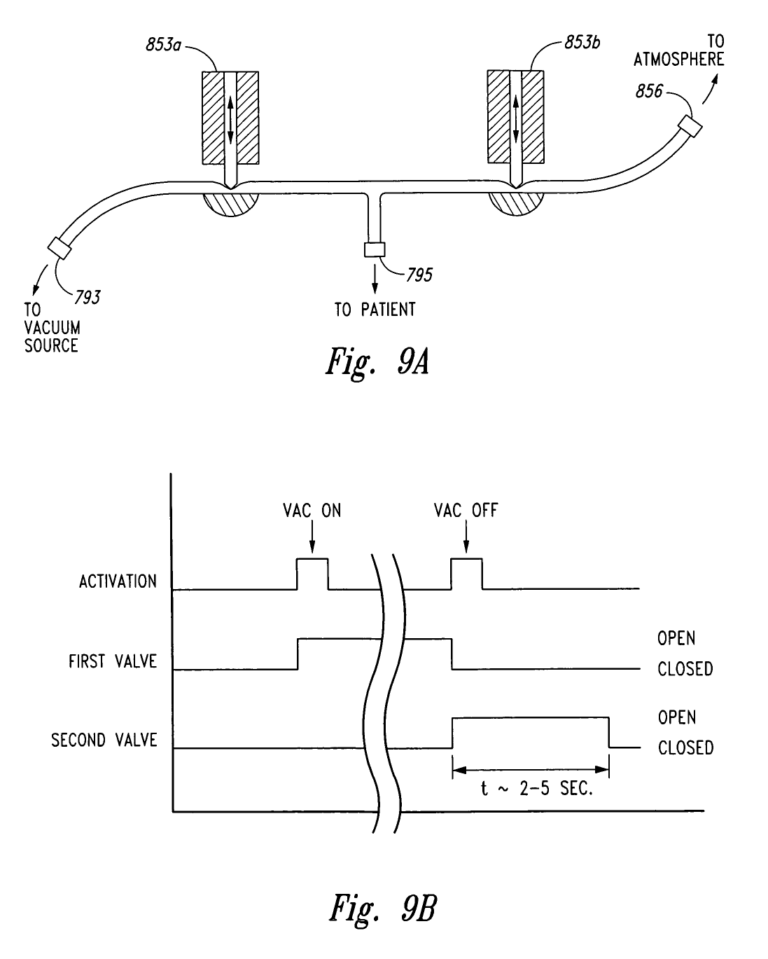

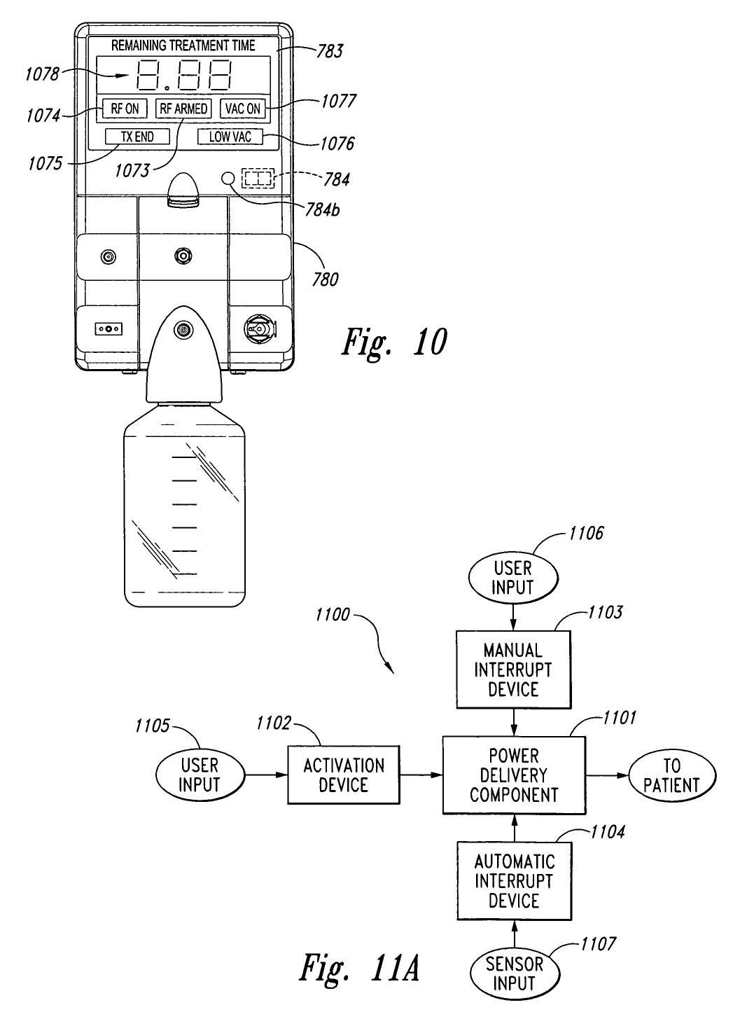

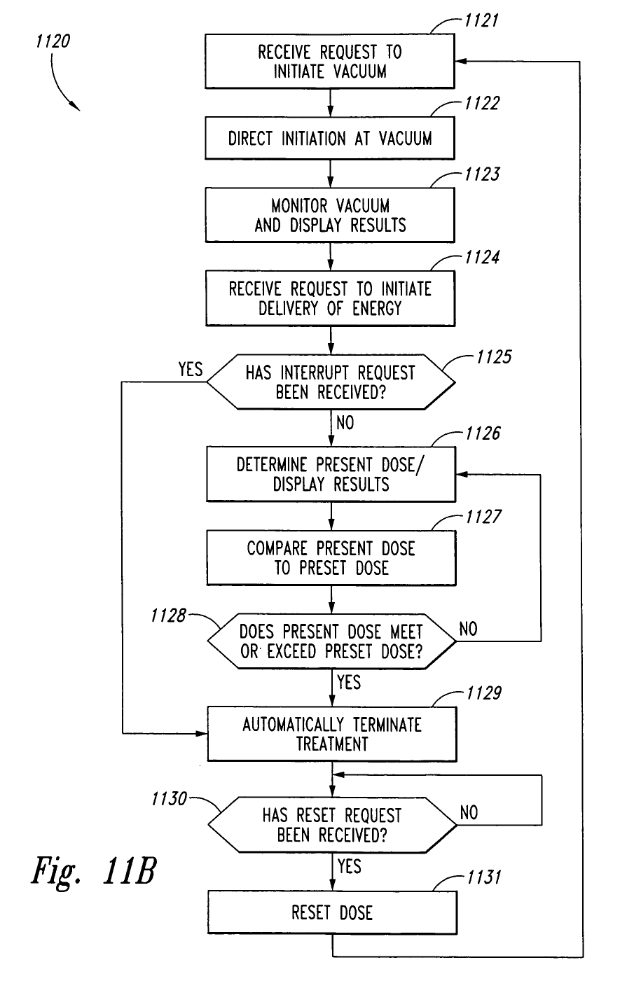

FIG. 6J illustrates an inflatable member configured to carry a recirculating fluid in accordance with still another embodiment of the invention.

FIG. 6K illustrates a working portion having a heat sink configured in accordance with an embodiment of the invention.

FIGS. 7A-7C illustrate a console and disposable collection unit configured in accordance with an embodiment of the invention.

FIGS. 8A-8B illustrate further aspects of an embodiment of the disposable collection unit shown in FIG. 7A.

FIGS. 9A-9B schematically illustrate control valve operations in accordance with an embodiment of the invention.

FIG. 10 is an illustration of a display portion of a console configured in accordance with an embodiment of the invention.

FIG. 11A is a block diagram illustrating components of a control system in accordance with an embodiment of the invention.

FIG. 11B is a flow diagram illustrating operation of a catheter control system in accordance with still another embodiment of the invention.

FIG. 11C is a flow diagram illustrating operation of a catheter control system in accordance with yet another embodiment of the invention.

FIG. 12 is a partially schematic illustration of a liquid collection vessel configured in accordance with another embodiment of the invention.

### DETAILED DESCRIPTION

A. Introduction

Aspects of the present invention are directed generally to methods and devices for drawing portions of cardiovascular tissue together, sealing the portions to each other, and controlling the performance of these tasks. For example, a device for treating a patent foramen ovale (PFO) in accordance with one aspect of the invention includes a catheter having a proximal end and a distal end. The catheter can include a working portion that is positioned toward the distal end, and is elongated along a terminal axis. An energy transmitter (e.g., an electrode) is positioned at the working portion of the catheter and can be tapered inwardly toward the terminal axis in a distal direction, asymmetrically relative to the terminal axis. For example, the energy transmitter can have an asymmetrical conical shape. The energy transmitter can also include an external surface with a concave recess, for example, a recess that can be positioned to engage with a portion of the tissue at or near the PFO. The energy transmitter can include one or more vacuum apertures coupleable to a vacuum source, for example, to aid in drawing the adjacent cardiac tissue into contact with the energy transmitter. The device can also include a heat sink positioned to transfer heat away from the energy transmitter.

In still further embodiments, the electrode or other energy transmitter can have an internal guidewire conduit that is non-parallel to the terminal axis and that slideably receives a guidewire. In yet another embodiment, the working portion of the catheter can have a non-zero bend angle relative to an adjacent portion of the catheter. The electrode and the bend angle can be positioned relative to each other so that the electrode is symmetric relative to a plane that also includes the bend angle. These and other arrangements and alignments of the portions of the device can, in at least some cases, aid the practitioner in aligning the device in the patient and providing a secure seal between the device and the adjacent cardiac tissue. For example, the device can also include an asymmetrically shaped inflatable member (e.g., a balloon) that provides a seal between the cardiac tissue and the working portion of the catheter.

Particular aspects are also directed to methods for treating a PFO located between a septum primum and a septum secundum of the patient. One such method can include positioning a working portion of a catheter proximate to the PFO, with the working portion elongated along a terminal axis. The method can further include engaging an energy transmitter (e.g., an electrode) carried by the working portion with tissue adjacent to the PFO by contacting a first surface of the energy transmitter with the septum primum and engaging a second surface of the energy transmitter with the septum secundum, while the first and second surfaces have different angular orientations relative to the terminal axis. In further particular embodiments, a vacuum can be drawn through the energy transmitter to draw the septum secundum and the septum primum toward the energy transmitter.

B. Catheters and Associated Methods for Treating Cardiac Tissue

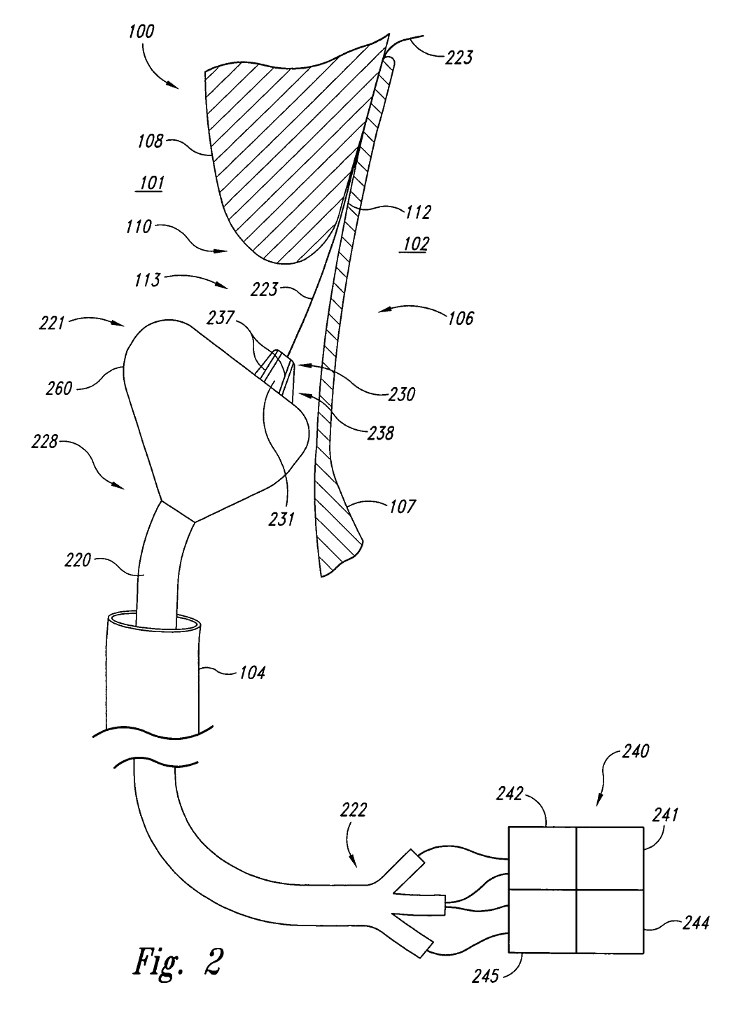

FIGS. 2-5B illustrate a catheter 220 and methods for using the catheter 220 to treat cardiovascular tissue, in accordance with several embodiments of the invention. These Figures, as well as FIGS. 6A-6K and the associated discussion, illustrate implementations of representative devices and methods in the context of cardiac tissues. In other embodiments, at least certain aspects of these devices and methods may be used in conjunction with other tissues, including other cardiovascular tissues (e.g., veins or arteries).

Beginning with FIG. 2, the catheter 220 can include a proximal end 222 coupled to a control unit 240, and a distal end 221 having a working portion 228 configured to be placed in a patient's heart 100. At least part of the catheter 220 can be flexible so as to allow the catheter 220 to absorb stresses without disturbing the working portion 228. The distal end 221 of the catheter 220 can be inserted into the patient's heart 100 via the inferior vena cava 104 or another blood vessel, and can be threaded along a guidewire 223. The catheter 220 can include a vacuum system 238 having vacuum ports 237 that are used to evacuate fluids (and/or solids, e.g., blood clots) in the region surrounding the distal end 221. The vacuum ports 237 can have a slot shape as shown in FIG. 2, or other shapes in other embodiments. The force of the applied vacuum can draw portions of the cardiac tissue toward each other and toward the catheter 220.

The catheter 220 can also include an energy transmitter 230 (e.g., an electrode 231) that directs energy (e.g., RF energy) to the cardiac tissue portions to bond the tissue portions together. Much of the following discussion references an energy transmitter 230 that includes the electrode 231, but in other embodiments, the energy transmitter can include other devices and/or devices that transmit other forms of energy (e.g., ultrasonic energy or laser energy). Any of these devices may generate heat that, in addition to fusing the tissue together, may cause the tissue to adhere to the catheter 220. Accordingly, in at least some embodiments, an optional fluid supply system can provide fluid to the working portion 228 to prevent the cardiac tissue from fusing to the electrode 231 or other portions of the energy transmitter 230, and/or to increase the penetration of the electrical field provided by the electrode 231. Details of the fluid supply system are not shown in FIG. 2, but are described in greater detail in U.S. Provisional Application 60/727,678, previously incorporated herein by reference.

The working portion 228 can also include an inflatable member 260 (e.g., a balloon, sack, pouch, bladder, membrane, circumferentially reinforced membrane, or other suitable device) located proximate to the electrode 231. The inflatable member 260 can be selectively deployed and inflated to aid in releaseably sealing the catheter 220 at or proximate to the target tissue to which energy is directed. When the inflatable member 260 is inflated, the electrode 231 can project from the inflatable member 260 in a distal direction so that the electrode 231 is in intimate contact with the target tissue.

The control unit 240 can control and/or monitor the operation of the inflatable member 260, the energy transmitter 230, and the vacuum system 238. Accordingly, the control unit 240 can include an inflatable member controller 245, an energy transmitter control/monitor 241, and a vacuum control/monitor 242. The control unit 240 can also include other controls 244 for controlling other systems or subsystems that form portions of, or are used in conjunction with, the catheter 220. Such subsystems can include, but are not limited to, the fluid supply system described above, and/or temperature and/or impedance detectors that determine the temperature and/or impedance of the cardiac tissue and can be used to prevent the energy transmitter 230 from supplying excessive energy to the cardiac tissue. The subsystems can also include current sensors to detect the current level of electrical signals applied to the tissue, voltage sensors to detect the voltage of the electrical signals, and/or vision devices that aid the surgeon or other practitioner in guiding the catheter 220. The control unit 240 can include programmable, computer-readable media, along with input devices that allow the practitioner to select control functions. The control unit 240 can also include output devices (e.g., display screens) that present information corresponding to the operation of the catheter 220. Further details regarding several of the foregoing features are described later with reference to FIGS. 7A-12.

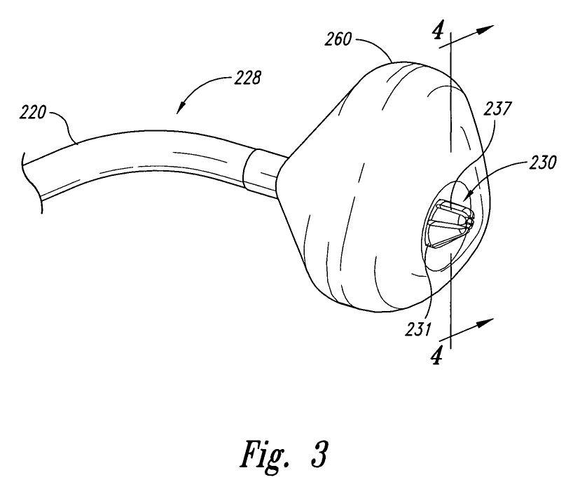

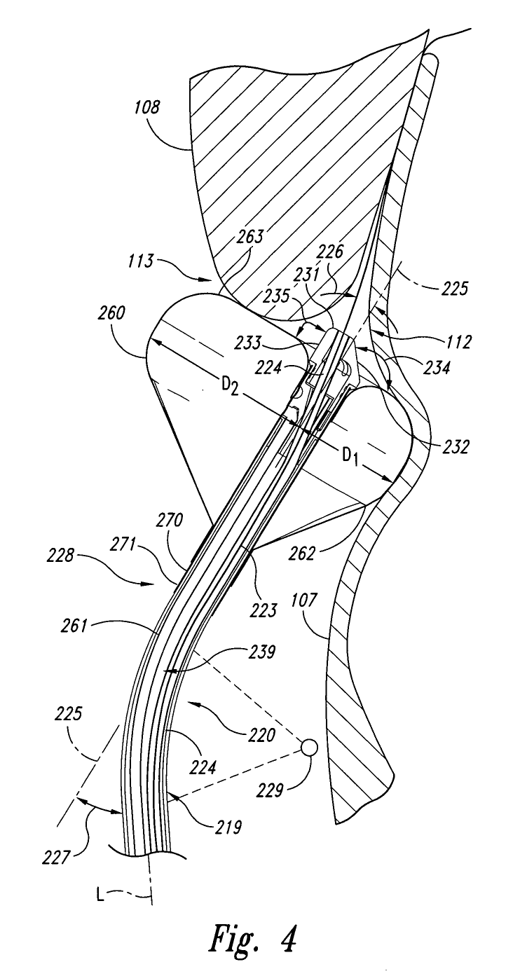

FIG. 3 is an enlarged, isometric illustration of the working portion 228 of the catheter 220 shown in FIG. 2. As shown in FIG. 3, the inflatable member 260 can have a roughly triangular or pear-like shape when viewed head-on that, in at least some cases, is roughly similar to the shape of the fossa ovalis. It is expected that the shape of the inflatable member 260 will facilitate sealing the inflatable member 260 against the septal tissue, while the electrode 231 projects away from the inflatable member 260 to extend at least part way into the PFO, with the vacuum ports 237 exposed. Particular aspects and combinations of aspects of the features shown in FIGS. 2 and 3 are described in greater detail below with reference to FIG. 4.

FIG. 4 is a partial cross-sectional illustration of the working portion 228 of the catheter 220, positioned proximate to a PFO 113, and taken generally along line 4-4 of FIG. 3. The working portion 228 is elongated generally along a terminal axis 225. The electrode 231 and/or the inflatable member 260 can be asymmetric relative to the terminal axis 225. An expected benefit of this arrangement is that it can allow for an improved seal between the working portion 228 and the adjacent cardiac tissue, and/or improved energy delivery from the electrode 231 to the tissue.

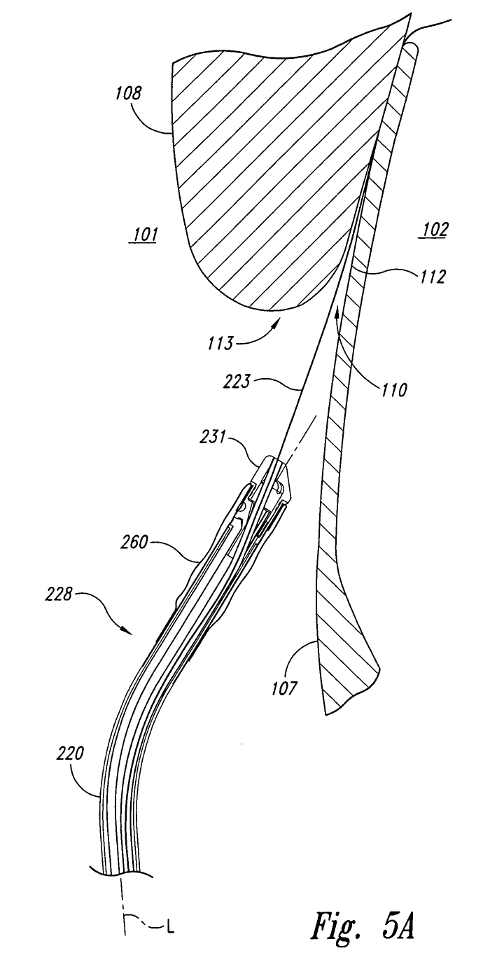

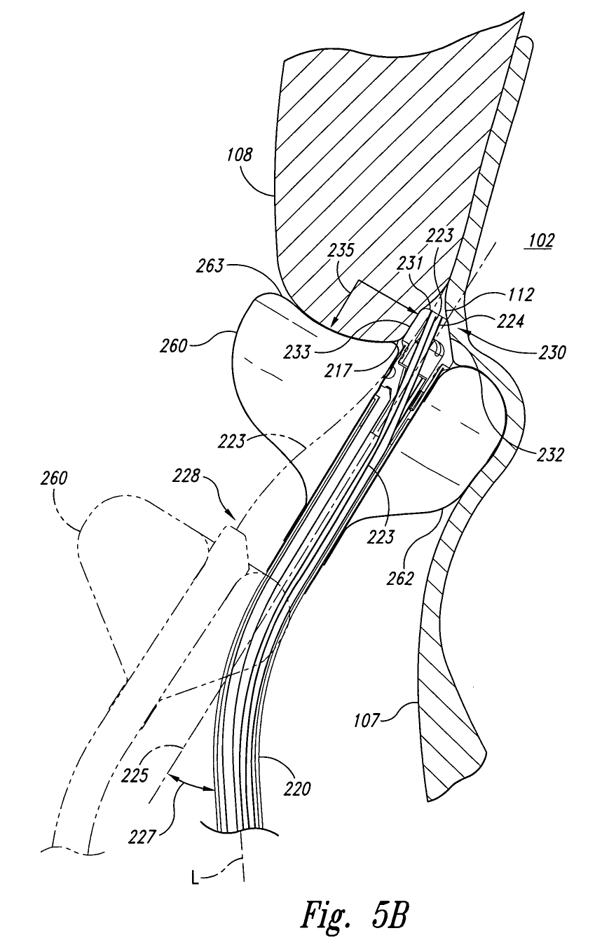

In a particular embodiment, the inflatable member 260 can include a first inflatable portion 262 (e.g., an inferior portion) and a second inflatable portion 263 (e.g., a superior portion) that extend by different distances from the terminal axis 225. In particular, the first inflatable portion 262 can extend away from the terminal axis 225 by a distance D1 that is less than a distance D2 by which the second inflatable portion 263 extends away from the terminal axis 225. A representative value for D1 is about 8 mm. Accordingly, a greater portion of the inflatable member 260 can contact the secundum 108 then the primum 107. As will be described in greater detail below with reference to FIGS. 5A-5B, this arrangement can take advantage of the more robust structure of the secundum 108.

The inflatable member 260 can be constructed from a compliant urethane material (e.g., having a durometer value of from about 50 to about 80 on the Shore A scale). One such material includes Pellethane®, available from the Dow Chemical Company of Midland, Mich. This material can be readily bonded to the shaft of the catheter 220 thermally or adhesively, and can be selected to be translucent or transparent, allowing the practitioner to view a fluid contrast agent that may be used to inflate the inflatable member 260. The material forming the inflatable member 260 can also be selected to be quite compliant so as to conform to the tissue against which it temporarily seals, without displacing or distorting the tissue by a significant amount. Such compliancy can also make the inflatable member 260 easier to stow aboard the catheter 220, as the catheter is introduced into the patient's body (prior to inflation), and as the catheter is removed from the patient's body (after inflation and treatment). The material forming inflatable member 260 can be thin (e.g., 25-50 microns thick) to facilitate compliancy. In particular embodiments, the material forming the inflatable member 260 can be thicker at some portions than at others, to produce the desired shape after inflation. For example, the most distal face and/or perimeter sections of the inflatable member 260 may be constructed to be thinner than other portions of the inflatable member 260. When inflated with a liquid, this thin portion may more readily take a rounded shape and will remain compliant, so as to assist in providing improved sealing under vacuum, and/or assist in placing the electrode 231 at a selected axial position inside the PFO tunnel 112. Further details of such an arrangement are described later with reference to FIG. 6H.

The inflatable member 260 can be inflated with any suitable fluid, including saline. The fluid can also include a contrast agent to aid the practitioner in locating the inflatable member 260 relative to other structures. In particular embodiments, the contrast agent can include MD-76®R or Optiray® 320 available from Mallinckrodt, Inc. of St. Louis, MO. The contrast agent can be diluted to reduce its viscosity and therefore increase the rate with which the inflatable member 260 is inflated and deflated. For example, the inflation fluid can include 10-50% contrast agent (the remainder being saline), with 25% or 50% contrast agent in particular embodiments. With fluid compositions having these characteristics, a representative inflatable member 260 carried by a representative catheter 220 (e.g., one having an internal diameter of 0.025-0.28 inches) can be fully inflated in 10-15 seconds or less.

The electrode 231 can also be asymmetric relative to the terminal axis 225. For example, the electrode 231 can include a first electrode portion 232 (e.g., an inferior portion) and a differently shaped second electrode portion 233 (e.g., a superior portion). The first electrode portion 232 can form a first electrode angle 234 relative to the inflatable member 260, and the second electrode portion 233 can form a second, different electrode angle 235 relative to the inflatable member 260. For example, the second electrode angle 235 can be approximately 90° (so that the superior surface is generally parallel to the terminal axis 225), while the first electrode angle 232 can have a value other than 90°. In a particular embodiment, the first electrode angle 234 can have a value of about 147°, corresponding to an acute angle relative to the terminal axis 225 of about 33°. In other embodiments, the first electrode angle 234 can have other values, e.g., other values greater than 90°. Such angles can include angles in the range of from about 130° to about 160°, corresponding to acute angles relative to the terminal axis 225 of from about 50° to about 20°.

As a result of the foregoing arrangement, the first electrode portion 232 can have a conical shape with a relatively large external surface area, which can increase the efficiency with which the adjacent cardiac tissue is heated during the tissue welding operation. The taper angle of the first electrode portion 232 may also aid in directing the RF energy emitted from the electrode 231 directly into the PFO tunnel 112 to more efficiently weld this tissue. The presence of the inflatable member 260 (which is generally, if not entirely non-conductive) can also act to direct RF energy forward into the tissue immediately adjacent to the PFO tunnel 112. In addition, the taper angle of the first electrode portion 232 can more accurately align this portion of the electrode 231 with the natural orientation of the adjacent primum 107. The relatively short axial length of the electrode 231 can (a) reduce the extent to which the electrode 231 displaces the primum 107, and/or (b) allow the electrode 231 to be placed in relatively short PFO tunnels 112, while still providing effective PFO sealing.

In a particular embodiment, the electrode 231 can be manufactured from 17-4 stainless steel or an equivalent electrically conductive, bio-compatible material including, but not limited to platinum or platinum iridium. These materials can be suitable for conducting RF energy, and also for machining small features (e.g., the vacuum ports 237 shown in FIG. 3). These materials are also relatively easy to bond to the shaft and/or associated shaft components of the catheter 220.

In operation, it is typically desirable to seal the PFO 113 as quickly as possible so as to minimize the invasiveness of the procedure. However, if electrical energy is delivered too aggressively (e.g., via too high a current level), the adjacent tissue may bond or stick to the electrode 231. When the electrode 231 is later removed from the patient, it can disrupt or de-bond the tissue weld. High current can also create local “hot spots” that can result in potentially damaging eruptions of steam. In addition, the impedance of the tissue adjacent to the electrode 231 can increase rapidly when heated, which in turn reduces the penetration of the RF energy emitted by the electrode. This “impeding out” effect can therefore reduce the extent and strength of the resulting tissue seal. On the other hand, if the current density is reduced by reducing the applied current, the welding process can take longer to perform. If the current density is reduced by increasing the electrode size, the electrode diameter may become too large to be easily introduced into the patient, and/or may unnecessarily heat adjacent tissue.

To address the foregoing effects, the catheter 220 can include a heat transfer element (e.g., a heat sink) 270 that is in thermal communication with the electrode 231 and, in an embodiment shown in FIG. 4, extends in a proximal direction along the catheter 220 away from the electrode 231. The heat sink 270 can be electrically insulated from its surroundings, for example, via a thin, thermally conductive, but electrically insulating film or coating 271 that can include Teflone® or another biocompatible material. The coating 271 can have a sleeve shape to fit over the heat sink 270, with a representative thickness of 1-10 microns, and a representative thermal resistance of 2° C./watt or less. The heat sink 270 can also be formed from a material having a relatively high thermal conductivity, such as silver or a silver alloy. In other embodiments, the heat sink 270 can be formed from copper, gold, or alloys of these metals, or plated-on combinations of metals. For example, in a particular embodiment, the heat sink 270 is formed from a gold plated, silver-copper alloy. The gold plating provides a good interface with the adjacent cardiac tissue, and the silver-copper alloy (e.g., approximately 90% silver and approximately 10% copper in a representative embodiment) provides high thermal and electrical conductivity, combined with good material strength and machinability. In a particular embodiment, the gold plating can have a thickness of from about 2 microns to about 20 microns (e.g., about 5 microns) and in other embodiments, the plating thickness can have other values. The heat sink 270 can be formed integrally with the electrode 231 (e.g., the heat sink 270 and the electrode 231 can be machined or cast or otherwise formed from a single piece of metal stock), or the heat sink 270 can be an initially separate component that is placed in intimate, contiguous thermal contact with the proximal surface of the electrode 231. In either arrangement, the heat sink 270 can have a generally cylindrical shape with internal openings to accommodate vacuum channels, inflation channels and/or electrical leads. Accordingly, the outer surface of the heat sink 270 can be positioned in thermal contact with and adjacent to the inner annular surface of the inflatable member 260 and also the fluid within the inflatable member 260. As a result, the heat sink 270 can transfer heat from the electrode 231 to the fluid within the inflatable member 260.

Heat can readily transfer from the heat sink 270 into the fluid within the inflatable member 260. Furthermore, because the material forming the inflatable member 260 is quite thin, heat can readily transfer from the fluid inside the inflatable member 260 to the surrounding blood and/or tissue. The fluid within the inflatable member 260 is expected to circulate throughout the inflatable member 260 due to convection resulting from the heat supplied by the heat sink 270 and/or the electrode 231, and/or due to mechanical agitation produced by the beating heart in which the inflatable member 260 is positioned.

In particular embodiments, the heat sink 270 can extend in a proximal direction beyond the inflatable member 260, as shown in FIG. 4. Accordingly, the heat sink 270 can be cooled directly by the circulating blood, as well as indirectly by the fluid in the inflatable member 260. In other embodiments, the heat sink 270 can be cooled solely by either direct or indirect heat transfer. The arrangement of the heat sink 270, the inflatable member 260, and the electrode 231 provides a low thermal resistance pathway for heat to be conveyed away from the electrode 231 and the immediately adjacent tissue. In still further embodiments, heat can be transferred away from the electrode 231 in accordance with related techniques, including those disclosed in U.S. Pat. No. 4,492,231, incorporated herein by reference.

In still further embodiments, other techniques can be used to reduce or eliminate sticking between the tissue and the electrode 231, in addition to or in lieu of transferring heat with the heat sink 270. For example, the voltage applied to the electrode 231 can be limited to a particular range. In some cases, when tissue desiccation occurs at the interface between the electrode 231 and the adjacent tissue, the electric field strength tends to increase. This can result in voltages high enough to achieve ionization or arcing in the liquid (or in some cases, gas) between the tissue and the electrode surface. Accordingly, in at least some embodiments, the maximum voltage provided by the system may be clamped or capped, for example, at 50 volts rms.

In operation, it is expected that the heat sink 270 can transfer heat from the electrode 231 at a rate sufficient to prevent or at least reduce sticking between the electrode 231 and the adjacent cardiac tissue. For example, the heat sink 270 is expected to transfer heat from the electrode 231 rapidly enough to keep the electrode 231 within 6° C. of the patient's body temperature, in at least one embodiment, and within 4° C. of the patient's body temperature in a further particular embodiment. The interface between the electrode 231 and the adjacent cardiac tissue is expected to experience a limited temperature increase of 10° C. or less, per watt of energy removed by the heat sink 270 (e.g., in an aft or proximal direction away from the electrode 231 and/or away from the adjacent cardiac tissue). For example, the temperature increase may be about 2° C. per watt of removed heat energy, with the amount of removed heat energy at a level of about one watt. At the same time, the amount of thermal energy applied to the adjacent tissue can be about 10 watts. It is expected that this arrangement will allow tissue sealing to within a very close distance of the electrode 231, without causing the tissue to adhere to the electrode 231 itself. For example, the secundum 108 and the primum 107 can seal to each other beyond a distance of about 0.3 mm. from the electrode 231. It is also expected that transferring heat from the electrode 231 will reduce the rate at which the adjacent cardiac tissue experiences a significant impedance increase as it is heated and welded. An expected benefit of this arrangement is that the RF energy can penetrate deeper into the PFO tunnel 112 (lengthwise and/or widthwise) before the increase in impedance inhibits the transmission of RF energy. As a result, the seal between the primum 107 and the secundum 108 is expected to be more extensive, more complete and/or more robust than it otherwise would be. In particular, for larger PFOs, deeper penetration with more energy delivered in both a lengthwise and a widthwise direction can provide for a broader tissue seal with an increased seal surface area.

The working portion 228 of the catheter 220 can also include a guidewire conduit or lumen 224 that extends through the electrode 231. The guidewire conduit 224 slideably receives the guidewire 223 over which the catheter 220 is introduced into the heart. The guidewire conduit 224 can also control the path of the guidewire 223 relative to the catheter 220. As is shown in FIG. 4, the distal portion of guidewire conduit 224 can be oriented at a non-zero path angle 226 relative to the terminal axis 225. In a particular aspect of this embodiment, the guidewire conduit 224 can be oriented so that the path angle 226 is approximately 9°. In other embodiments, the path angle 226 can have other values (e.g., in the range of from about 3° to about 20°). As a result of this construction, the guidewire 223 will be oriented obliquely relative to the terminal axis 225. This arrangement can more accurately align the axis of the guidewire 223 with the axis of the PFO tunnel 112 into which the guidewire 223 is inserted. As a result, the guidewire 223 is expected to be less likely to push, “tent” or otherwise displace the primum 107 away from the secundum 108, which augments the RF treatment/welding process.

The remainder of the generally hollow interior portion of the catheter 220 can operate as a vacuum lumen 239. Accordingly, the vacuum lumen 239 can have a relatively large cross-sectional area transverse to the terminal axis 225 to efficiently draw a vacuum through the catheter 220. When coupled to a vacuum source, the vacuum lumen 239 can provide a vacuum to the vacuum ports 237 (FIG. 3) to draw the septal tissue into contact with the electrode 231. In a particular embodiment, the catheter 220 can be constructed from a reinforced, braided material to resist collapsing under vacuum.

The catheter 220 can include a catheter bend 219 positioned so that the terminal axis 225 is offset relative to a longitudinal axis L of the immediately adjacent portion of the catheter 220. The bend 219 can be pre-formed into the catheter 220, but the catheter 220 can be flexible enough so that as it is inserted through an introducer sheath and threaded along the guidewire 223 (e.g., through the femoral vein), it will tend to straighten out. Once it enters the less constrained volume within the heart, the catheter 220 can assume its bent configuration. In a particular embodiment, a bend angle 227 between the terminal axis 225 and the longitudinal axis L can have a value of about 45°, and in other embodiments, the bend angle 227 can have other values. For example, the bend angle 227 can have a value in the range of from about 20° to about 90° in one embodiment, and from about 30° to about 80° in another embodiment. The catheter 220 can also be bent relatively uniformly (e.g., at a generally constant and relatively small radius) relative to a center of curvature 229 located in the plane of FIG. 4. In particular embodiments, the bend angle 227 can be adjustable by the practitioner. For example, the catheter 220 can include one or more cables or other control features (not shown in FIG. 4) that the practitioner can manipulate to adjust the value of the bend angle 227 and improve the practitioner's ability to accurately position the electrode 231 and the inflatable member 260. In a particular embodiment, the practitioner can use a steerable introducer sheath or a steerable outer catheter to aid in positioning the electrode 231 and the inflatable member 260.

The bend angle 227, the guidewire exit angle 226, and the first electrode angle 234 can have deliberately selected orientations relative to each other. For example, the bend angle 227, the guidewire exit angle 226, and the first electrode angle 234 can all be located in the same plane (e.g., the plane of FIG. 4). The maximum amount by which the first inflatable portion 262 extends from the terminal axis 225 (e.g., D1) and the maximum amount by which the second inflatable portion 263 extends from the terminal axis 225 (e.g., D2) can also be located in the plane of FIG. 4. Accordingly, the generally flat superior surface of the electrode 231 and the apex of the inflatable member 260 can face in one direction, while the tapered surface of the electrode 231 and the base of the inflatable member 260 can face in the opposite direction. As a result of this orientation, the working portion 228 (including the electrode 231, the inflatable member 260, and the guidewire conduit 224) can all be symmetric relative to the plane of FIG. 4, although these components are asymmetric relative to the terminal axis 225. As will be described below with reference to FIGS. 5A-5B, providing a known relationship between the foregoing angles and orientations can improve the accuracy with which the practitioner aligns the working portion 228 prior to a PFO sealing procedure, particularly when a significant axial pressure may be applied to the catheter 220 to aid in sealing the working portion 228 to the adjacent tissue.

FIGS. 5A-5B illustrate the operation of the catheter 220 in accordance with an embodiment of the invention. Beginning with FIG. 5A, the catheter 220 is inserted into the right atrium 101 to seal a PFO 113 between the right atrium 101 and the left atrium 102. Accordingly, the practitioner can first pass the guidewire 223 into the right atrium 101 and through the tunnel portion 112 of the PFO 113 using one or more suitable guide techniques. For example, the guidewire 223 can be moved inferiorally along the interatrial secundum 108 until it “pops” into the depression formed by the fossa ovalis 110. This motion can be detected by the practitioner at the proximal end of the guidewire 223. The tunnel 112 is typically at least partially collapsed on itself prior to the insertion of the catheter 220, so the practitioner will likely probe the fossa ovalis 110 to locate the tunnel entrance, and then pry the tunnel 112 open. Suitable imaging/optical techniques (e.g., fluoroscopic techniques, intracardiac echo or ICE techniques, and/or transesophageal electrocardiography or TEE can be used in addition to or in lieu of the foregoing technique to thread the guidewire 223 through the tunnel 112. Corresponding imaging/optical devices can be carried by the catheter 220.

Once the guidewire 223 has been inserted through the PFO 113 and into the left atrium, the catheter 220 is passed along the guidewire 223. The inflatable member 260 is initially in its collapsed state, as shown in FIG. 5A. The inflatable member 260 may include pleats and/or other features that allow it to fold neatly and compactly along the catheter 220 so as to fit through existing introducer sheaths as the catheter 220 is inserted into the body.

The practitioner may in some instances wish to use the inflatable member 260 to help determine the size and/or geometry of the PFO tunnel 112. Representative features of interest to the practitioner include the diameter, length, entrance shape and/or angle of the PFO tunnel 112. In one process, the practitioner inserts the working portion 228 into the PFO tunnel 112 until the inflatable member 260 is within the tunnel 112. Using a suitable visualization technique (e.g., ICE or fluoroscopy), the practitioner can then slowly and/or incrementally inflate the inflatable member 260 until the inflation is constrained by the primum 107 and/or the secundum 108. Even though the primum 107 and the secundum 108 may not be readily visible (as they may not be during fluoroscopy visualization), the inflated inflatable member 260 will be visible. By measuring the size of the inflatable member 260 (at one or more locations) on a display monitor, and scaling this dimension relative to the known diameter of the working portion 228, the practitioner can estimate the size of the tunnel 112. This information can help the practitioner determine treatment parameters, including how far to insert the electrode 231, how to position the inflatable member 260, how much forward pressure to apply to the inflatable member 260, how much to inflate the inflatable member 260, and/or how much energy to deliver with the electrode 231.

If the inflatable member 260 is used to size the tunnel 112, it can then be deflated and withdrawn from the tunnel 112 into the right atrium 101. Once the catheter 220 is in the right atrium 101, the inflatable member 260 is inflated, as is shown in broken lines in FIG. 5B, and the inflatable member 260 may now be used to provide the additional function of sealing the interface between the catheter 220 and the adjacent cardiac tissue. The practitioner can rotate the catheter 220 about its longitudinal axis L until the catheter 220 is at the desired orientation. In an embodiment such as that described above with reference to FIG. 4, in which the asymmetric features of the working portion 228 are all aligned, the practitioner can adjust the position of one such feature, and know that the remaining features will also be aligned. For example, in some cases, the bend angle 227 of the catheter 220 may be the feature most visible to the practitioner. In other cases, the inflatable member 260 may be the most visible. In either case, the practitioner can align one feature (e.g., the most readily visible feature) with an individual patient's cardiac landmarks, and know that other features (e.g., the electrode 231) will have a known, proper orientation.

When the catheter 220 is properly oriented, it is advanced along the guidewire 223 until the electrode 231 extends just inside the PFO tunnel 112, and the inflatable member 260 (generally having the shape indicated by broken lines in FIG. 5B), contacts the secundum 108 and the primum 107. At this point, the practitioner can apply an axial force to the catheter 220, causing the inflatable member 260 to bear against the secundum 108 and the primum 107. Because the secundum 108 is relatively robust, it tends to cause the second inflatable portion 263 of the inflatable member 260 to deform, as indicated in solid lines in FIG. 5B. Because the primum 107 is more compliant, it tends to react to the axial and circumferential pressure by conforming around the first inflatable portion 262, as is also shown in solid lines in FIG. 5B. The guidewire 223 can remain in position in the PFO tunnel 112 during this phase of the process. At this point, the vacuum system can be activated to draw a vacuum through the vacuum ports 237 (FIG. 3) of the electrode 231, drawing the secundum 108 and the primum 107 against the electrode 231 and the inflatable member 260, and removing blood and/or other fluids from the treatment site.

The practitioner can use any of several techniques to determine when the proper seal between the working portion 228 and the adjacent tissue is achieved, and/or to determine how to make adjustments, if necessary. For example, the practitioner can receive at least a gross indication of a proper seal by observing the shape of the inflatable member 260. When the inflatable member 260 assumes a shape generally similar to that shown in solid lines in FIG. 5B (visible via fluoroscopy, ICE, or another suitable visualization technique), the practitioner can receive an indication that the inflatable member 260 is in at least approximately the correct location, and/or that the proper axial pressure is being applied. The practitioner can also observe the rate at which blood or other fluid is withdrawn through the catheter 260, and can determine that the proper seal is achieved when the blood flow ceases or reaches a de minimis level. If the blood flow does not cease within the expected time frame, the practitioner can use the oxygenation level of the blood to determine the location of the leak. For example, if the withdrawn blood is deoxygenated, this may indicate that the leak is at the right atrium. If the blood is oxygenated, this may indicate that the leak is at the left atrium. For example, the presence of oxygenated blood may indicate that the PFO tunnel 112 is not fully collapsed, which may in turn indicate that the catheter 220 is propping the tunnel 112 open (e.g., if the catheter 220 is inserted too far into the tunnel 112). The practitioner can determine the oxygenation level of the blood by direct observation of the blood color, and/or by observing measurements from suitable devices, such as a pulse oximeter. Once the expected location of the leak is determined, the practitioner can adjust (e.g., reduce) the level of applied vacuum, re-position the catheter 220 and/or adjust the pressure of the inflatable member 260, and re-apply the vacuum until the proper seal is achieved.

Once the catheter 220 is securely held in position under the force of vacuum, the guidewire 223 can be pulled back into the catheter 220 so as not to extend into the PFO tunnel 112. At this time, the vacuum drawn on the cardiac tissue keeps the working portion 228 in a fixed position with the inflatable member 260 sealably positioned against the cardiac tissue. In at least some cases, the temporary vacuum seal between the catheter 220 and the adjacent cardiac tissue is strong enough to allow the practitioner to release his or her handhold on the catheter 220, allowing the practitioner the freedom to use his or her hands for other tasks. The energy transmitter 230 (e.g., the electrode 231) is then activated to heat the adjacent cardiac tissue and bond or at least partially bond the primum 107 and the secundum 108, thereby closing the PFO tunnel 112.

As shown in FIG. 5B, the asymmetry of the inflatable member 260 can allow for a greater portion of the inflatable member 260 to temporarily bear and seal against the secundum 108 than against the primum 107. An advantage of this feature is that the secundum 108 is generally more robust than the primum 107, and is expected to be better able to support the inflatable member 260 without undergoing a significant displacement, even if the practitioner applies an axial pressure to the catheter 220. As a result, the primum 107 can be less likely to be displaced away from the secundum 108 and/or the electrode 231 in a manner that may detract from the treatment process. Put another way, an alternate inflatable member that (a) is symmetric relative to the terminal axis 225, and (b) has the same surface area facing toward the PFO tunnel 112 as the inflatable member 260, may tend to extend inferiorly by a distance sufficient to push and/or stretch the primum 107 away from the secundum 108 and/or the electrode 231. An advantage of an embodiment of the inflatable member 260 shown in FIG. 5B is that it can reduce the extent to which the primum 107 is displaced or stretched, and can therefore increase the extent to which the primum 107 is tightly drawn against the electrode 231 and the secundum 108 during the tissue welding process. At the same time, the inflatable member 260 is configured to collapse down to a diameter that is small enough to allow use with readily available introducer sheaths (as shown in FIG. 5A).

The foregoing feature can be particularly appropriate for short PFO tunnels 112. It may be difficult to obtain a good seal between the inflatable member 260 and such tunnels because if the primum 107 is displaced, stretched, or distorted, the exit of the PFO tunnel 112 (in the left atrium 102) may open, causing the influx of fluid (blood) and inhibiting close contact between the secundum 108 and the primum 107. As described above, the asymmetrical shape of the inflatable member 260 can at least reduce the extent to which the primum 107 is displaced, stretched, or distorted in the region immediately adjacent to the PFO tunnel 112. Other shape features can also contribute to this effect. For example the relatively flat base of the inflatable member 260 allows the primum tissue to form a good seal with the inflatable member 260. In particular, the flat base may tend not to bulge away from the terminal axis, and accordingly may be less likely to displace the primum 107 away from the electrode 231. The asymmetrical shape of the inflatable member 260 can also increase accuracy of the alignment between the electrode 231 and the entrance of the PFO tunnel 112. This can in turn allow the RF energy to be directed more evenly into the PFO tunnel 112, rather than into the primum 107.

The pressure to which the inflatable member 260 is inflated can be relatively low in comparison to pressures typically used for angioplasty and other catheter balloons. For example, the inflatable member 260 can be inflated to a value of from 0.2 to 10 psi in one embodiment, and from 0.5 to 3 psi in a more particular embodiment. Pressure can be applied to the inflatable member 260 manually via a syringe filled with a liquid (e.g., a contrast agent), or automatically. The low pressures can be monitored with a suitable pressure gauge. These low pressures can further enhance the ability of the inflatable member 260 to conform to the local tissue topology and form a tight seal under vacuum. In operation, the practitioner can also apply axial pressure, and/or rotate the catheter 220 slightly clockwise or counterclockwise until a good seal is achieved. As discussed above, the fixed relative orientation of the various asymmetric features of the catheter 220 can reduce the extent to which the practitioner must make such adjustments.

In particular embodiments, the extent to which the inflatable member 260 is inflated can change the shape (as well as the size) of the inflatable member 260. For example, increasing the inflation pressure can increase axial length of the inflatable member 260, and therefore decrease the distance by which the electrode 231 projects forward of the inflatable member 260. This technique can be used to control the extent to which the electrode 231 penetrates into the PFO tunnel 112. The greater the inflation pressure, the more the inflatable member 260 tends to expand forwardly toward the electrode 231, and the shorter the distance by which the electrode 231 will penetrate into the PFO tunnel 112. In other embodiments, the inflation pressure applied to the inflatable member 260 can be used to control the orientation of the electrode 231. For example, at higher inflation pressures, the second portion 263 may tend to bulge forward more than does the first portion 262. As a result, when the inflatable member 260 is placed against the primum 107 and the secundum 108, it may tilt slightly counterclockwise (in the plane of FIG. 5B), inclining the electrode 231 toward the secundum side of the PFO tunnel 112. This motion can in turn align the guidewire 223 more with the secundum side of the PFO tunnel 112 than with the primum side, thereby reducing the tendency for the guidewire 223 to push or “tent” the primum 107 away from the electrode 231 and the secundum 108. As mentioned above, the primum 107 tends to be thinner than the secundum 108, and may therefore be more susceptible to “tenting,” in the absence of aligning the guidewire 223 along the secundum side of the PFO tunnel 112.

The orientation of the guidewire conduit 224 can supplement or in some cases replace the tilted orientation of the inflatable member 260 as a feature by which to orient the guidewire 223 along the secundum side of the PFO tunnel 112. For example, when the guidewire conduit 224 is inclined relative to the terminal axis 225 (as shown in FIG. 5B), the guidewire 223 will tend to exit the electrode 231 at an angle that is more accurately aligned with the naturally occurring angle of the PFO tunnel 112. As described above, an advantage of this feature is that the guidewire 223 will have a reduced tendency to push the relatively thin primum 107 away from the electrode 231 as the guidewire 223 is deployed into the PFO tunnel 112. Accordingly, the likelihood for tightly sealing the primum 107 against the electrode 231 and the secundum 108, and therefore providing a secure seal between the primum 107 and the secundum 108, can be significantly increased. In some embodiments, the guidewire 223 can be withdrawn from the PFO tunnel 112 during tissue sealing (as described above), and in other embodiments, the guidewire 223 can remain in the tunnel 112 during this process. In another embodiment, the guidewire 223 may remain in the tunnel for the initial portion of the treatment, and may be withdrawn during the delivery of RF energy.

FIG. 5B also illustrates the second electrode portion 233 bearing against the limbus 217 of the secundum 108. Because the second electrode angle 235 is approximately 90° rather than a significantly larger value, the electrode 231 will tend to “hook” upwardly against the limbus 217 rather than slide way from the limbus 217. Accordingly, once the electrode 231 is located at the entrance of the PFO tunnel 112, it will be less likely to be displaced (e.g., upwardly and to the left in FIG. 5B) during the application of forward pressure and the tissue welding operation. This arrangement can also allow the practitioner to more readily feel when the electrode 231 is properly seated at the entrance of the PFO tunnel 112. In other embodiments, this function can be achieved with an electrode 231 having a second electrode angle 235 with a value other than 90°. For example the second electrode angle can be in the range of about 80°-100° in one embodiment, and about 70°-110° in another embodiment. In still further embodiments, the superior surface of the electrode 231 can be concave (as described later with reference to FIG. 6E) to further enhance engagement with the limbus 217.

In an embodiment discussed above, the catheter bend angle 227 is located in a single plane, and is aligned with features of the inflatable member 260 and the electrode 231. As discussed above, this arrangement can allow the practitioner to position the inflatable member 260 and the electrode 231 based on the (perhaps more visible) bend in the catheter 220. In other embodiments, the catheter bend angle 227 need not be contained to a single plane, e.g., in cases where a multi-plane bend angle improves the practitioner's ability to position the inflatable member 260 and/or the electrode 231, and/or in cases where the inflatable member 260 and/or the electrode 231 are more visible to the practitioner than is the bend angle 227.

FIGS. 6A-6K illustrate catheter working portions having electrodes and/or inflatable members configured in accordance with still further embodiments of the invention. For example, FIG. 6A illustrates two representative working portions 62a, 628b, each with an offset curve shown in dashed lines in FIG. 6A, along with corresponding centers of curvature 629a, 629b. In each of these embodiments, the working portions 628a, 628bare curved about a corresponding center of curvature 629a, 629bthat is offset laterally from the center of curvature 229 initially shown in FIG. 4 and superimposed for purposes of illustration in FIG. 6A. In at least some cases (depending upon cardiac geometry), the offset center of curvature of the working portions 628a, 628b can improve the alignment of the inflatable member 260 and the electrode 231 relative to the PFO treatment site.

FIGS. 6B-6C illustrate a catheter 620b configured to house a deployable inner catheter, in accordance with another embodiment of the invention. Referring first to FIG. 6B, the catheter 620b can carry an electrode 631b in a stowed (e.g., more proximal) position. In this position, the electrode 631b has a spatial relationship relative to a corresponding inflatable member 660b that is generally similar to that shown in FIG. 4. FIG. 6C illustrates the electrode 631b after it has been deployed from the catheter 620b to a more distal position. The electrode 631b can be attached to an inner catheter 620c that is received within the outer catheter 620b for axial movement relative to the inflatable member 660b. In operation, the practitioner can deploy the electrode 631b by a selected distance relative to the inflatable member 660b, for example, to control the extent to which the electrode 631b penetrates the PFO tunnel. This technique can be used in addition to, or in lieu of, controlling the extent to which the inflatable member 660b is inflated, as described above with reference to FIG. 5B. An advantage of this particular embodiment is that the electrode 631b can keep the relatively thin primum 107 (FIG. 5B) from being pushed or “tented” away from the secundum 108 (FIG. 5B) in short PFO tunnels. In other embodiments, the catheter can include other arrangements that allow for relative motion between the electrode 631b and the inflatable member 660b. For example, the inflatable member 660b can be carried by a catheter that is axially movable relative to a catheter carrying the electrode 631 b.

The shape of the inflatable member 660b can be selected to correspond to the shape of the fossa ovalis or other relevant physiological feature. For example, if a particular patient or group of patients (human or non-human) has a fossa ovalis with a shape that is significantly different than the average shape, the practitioner can select an inflatable member with a corresponding mating shape. In a particular example shown in FIGS. 6B-6C, the inflatable member 660b can have a generally round shape, rather than the generally triangular shape shown in FIG. 6A. In another embodiment, shown in FIG. 6D, an inflatable member 660d can have a generally oval shape that is also expected to seal around the perimeter and interior of the fossa ovalis, in at least some embodiments, depending upon patient physiology. In other embodiments, the inflatable members can have other shapes that may depend upon the geometry of the particular fossa ovalis against which the inflatable members are intended to seal. In still further embodiments, the inflatable member can have a “generic” shape (e.g., round, oval, generally triangular) and can be so flexible that it readily conforms to different fossa ovale having a variety of different shapes. Accordingly, the practitioner can select a device having an inflatable member with a shape (e.g., perimeter shape, or distal portion shape) that generally reflects and/or conforms to the perimeter shape of the patient's fossa ovalis.

In certain embodiments, the inflatable member 660d need not be asymmetric relative to the terminal axis 225. For example, the inflatable member 660d can have an oval shape, as shown in FIG. 6D, but can be positioned symmetric relative to the terminal axis 225, so that the terminal axis 225 passes through the center of the inflatable member 660d. In other embodiments, the inflatable member can have another shape (e.g., a round shape) that may also be symmetric relative to the terminal axis 225. The shape, as well as the symmetry or lack of symmetry, can be selected by the practitioner based on the characteristics of the particular patient being treated, or other parameters.

FIG. 6E is a side elevation view of the catheter 620b carrying an inflatable member 660e configured in accordance with another embodiment of the invention. In one aspect of this embodiment, the inflatable member 660e is tilted relative to the terminal axis 225. Accordingly, an inflatable member tilt angle 659 between the inflatable member 660e and the terminal axis 225 has a value other than 90° (e.g., less than 90°). One result of this arrangement is that when the inflatable member 660e is positioned up against the primum 107 and secundum 108, the electrode 631b will be oriented more toward the secundum side of the PFO than the primum side of the PFO. As described above, this can reduce the tendency for the corresponding guidewire 623 to displace the primum 107, and can instead cause the guidewire 623 to track along the secundum side of the PFO tunnel. Another potential result of this arrangement is that the acute second electrode angle 635 between the electrode 63b and the inflatable member 660e can increase the tendency for the electrode 63b to hook the limbus 217, and provide intimate contact with the secundum 108. Additionally, this arrangement may allow for the more intimate contact between the electrode 631b and the adjacent tissue, resulting in a more efficient energy transfer to the tissue.

FIG. 6F is a side elevation view of an electrode 631f shaped in accordance with still another embodiment of the invention. In one aspect of this embodiment, the electrode 631f can include a second or superior portion 633 having a dished, concave and/or saddle-shaped superior surface 636. This shape can further increase the tendency for the electrode 631f to “hook” the limbus 217, and thereby improve the ability of the electrode 631f to remain in position during a tissue sealing procedure. This feature can also better resist axial pressure applied to the catheter by the practitioner. In particular, as the practitioner moves the catheter into the patient's body, the electrode 631f can tend to move upwardly against the limbus 217. The saddle shape of the superior surface 636 can prevent this force from dislodging the electrode 631f.

FIG. 6G illustrates the catheter 620b carrying an inflatable member 660g configured in accordance with another embodiment of the invention. The inflatable member 660g can include a forwardly facing first portion 662g and a rearwardly facing second portion 663g. The second portion 663g can include multiple ribs or other reinforcing members 664 that increase the stiffness of the second portion 663g relative to the first portion 662g. The ribs 664 can be formed integrally with the surface of the inflatable member 660g, or the ribs can be formed using other techniques, including adhesively attaching the ribs 664 after the inflatable member 660g has been formed. The ribs 664 can be located at the exterior surface of the inflatable member 660g, as shown in FIG. 6G, or at the interior surface. In at least some embodiments, the increased stiffness provided by the ribs 664 is expected to improve the ability of the inflatable member 660g to seal against the adjacent cardiac tissue by (a) providing enhanced support to the second portion 663g while (b) allowing the first portion 662g to flex in a conformal manner at the site of contact with the cardiac tissue and (c) resisting axial movement resulting from pressure imparted by the practitioner (discussed previously with reference to FIG. 6F).

FIG. 6H illustrates an inflatable member 660h having a first or forwardly facing inflatable portion 66h and a second or rearwardly facing inflatable portion 663h, each of which has a different stiffness in accordance with another embodiment of the invention. For example, the first inflatable portion 662h can be formed from a material having a lower durometer value than that of the second inflatable portion 663h. In another aspect of this embodiment, the thickness of the material forming the first inflatable portion 66h can be less than that of the material forming the second inflatable portion 66h. In still further embodiments, these features can be combined with each other and/or with other characteristics to produce different stiffnesses in each portion. Each inflatable portion 662h, 663h can include an attachment section 667 that is bonded to the corresponding catheter (not shown in FIG. 6H) using an adhesive or other bonding technique. The inflatable portions 662h, 663h can be connected to each other at a seam 666, for example, with an appropriate adhesive or weld (e.g., an RF weld). Each of the inflatable portions 662h, 663h can be blow-molded or formed in another suitable fashion. Such techniques are available from Interface Associates of Laguna Nigel, California and are also appropriate for forming inflatable members from a single element (e.g., without the seam 666).

One feature of the foregoing arrangement is that the first inflatable portion 662h can readily conform to the topology of the cardiac tissue, which can in turn provide for a good vacuum seal with the tissue. At the same time, the second inflatable portion 663h can have enough rigidity to maintain the overall shape of the inflatable member 660h even as the practitioner pushes the catheter and the inflatable member 660h in an axial direction to seal the inflatable member 660h against the cardiac tissue.

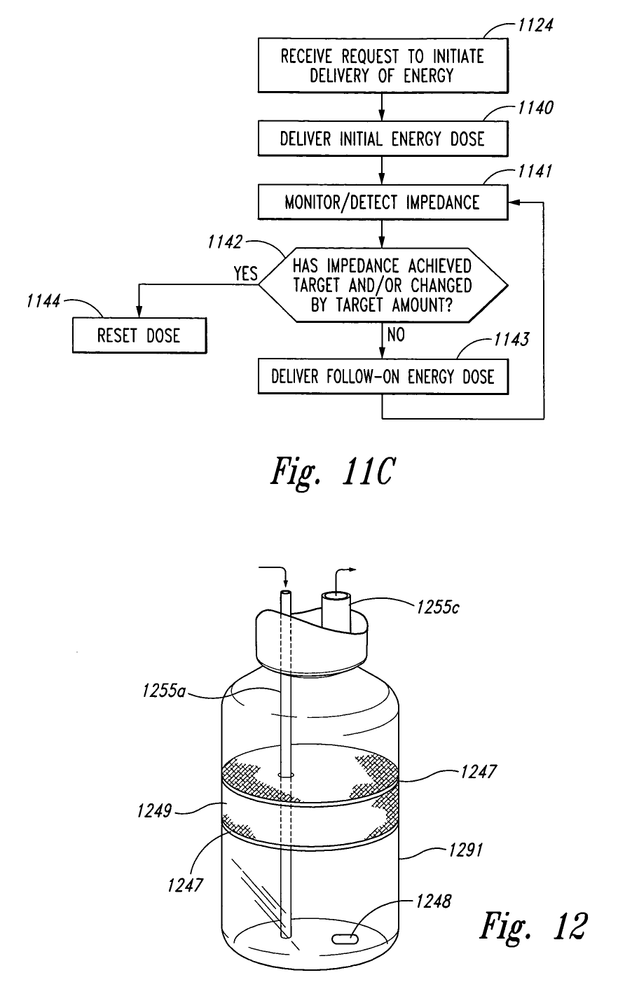

FIG. 61 illustrates a catheter 620i carrying an inflatable member 660i having two independently controllable inflatable chambers, including a first chamber 662i and a second chamber 663i. A chamber wall 665 separates the two chambers from each other. The catheter 620i can include separate first and second inflator lumens 661a, 661b, each with independent fluid communication with a respective one of the chambers 662i, 663i. Accordingly, the practitioner can control the shape, rigidity, and/or other characteristic of the inflatable member 660i by controlling the amount of pressure applied to each of the chambers 662i, 663i. For example, the practitioner can apply a relatively low pressure to the first chamber 662i, allowing the first chamber 662i to conform more readily to the adjacent cardiac tissue. At the same time, the practitioner can apply higher pressure to the second chamber 663i to provide for a more rigid support.

FIG. 6J illustrates another embodiment in which a recirculating fluid is used to inflate an inflatable member 660j. The first inflator lumen 661a can have a supply port 668a positioned in one region of the inflatable member 660j (e.g., toward the electrode 631b), and the second inflator lumen 661b can have a return port 668b located in another region of the inflatable member 660j (e.g., in a proximal direction from the electrode 631 b). Fluid is pumped into the inflatable member 660j via the supply port 668a, and returned via the return port 668b, as indicated by arrows J. The pressure and flow rate of the fluid can be controlled to control the extent to which the inflatable member 660j is inflated. Accordingly, in at least some embodiments, the inflatable member 660j can include an internal pressure transducer 669 that provides a feedback signal to allow the practitioner to monitor and control the inflation pressure. In another embodiment, the inflation pressure can be controlled automatically, based on the feedback signal. A temperature signal (e.g., provided by a thermocouple) can also provide an appropriate feedback mechanism. In any of these embodiments, the recirculating fluid in the inflatable member 660j can increase the rate at which heat is removed from the heat sink 270, and therefore the rate at which the electrode 631b is cooled. The recirculating fluid can also be directed into other system components, in addition to or in lieu of the inflatable member 660j. For example, the recirculating fluid can be cycled through the electrode 631b, provided the electrode 631b is outfitted with appropriate internal channels.

FIG. 6K is a partially exploded, partially cutaway illustration of an embodiment of the catheter working portion 228 initially described above with reference to FIG. 2. The working portion 228 can include the electrode 231 attached to the heat sink 270, which is in turn attached to a braided catheter shaft 603. The heat sink 270 can include one or more glue grooves 601 that retain a suitable adhesive for bonding the metallic heat sink 270 to the shaft 603. The heat sink 270 includes a vacuum lumen 639 (e.g., an integral, hollow center section) that aligns concentrically with the braided shaft 603, and couples to the vacuum ports 237 in the electrode 231. An inflator lumen 661 provides fluid to the inflatable member 260. The thin electrically insulating coating 271 (a portion of which is shown in FIG. 6K) allows for a high degree of thermal communication between the heat sink 270 and (a) fluid in the inflatable member 260 (directly, and through one of the inflatable member attachment sections 667a) and (b) to blood outside the inflatable member (directly, and through another of the inflatable member attachment sections 667b). As discussed above, heat transferred to fluid within the inflatable member 260 is then transmitted to the surrounding blood and tissue via the walls of the inflatable member 260.

The electrode 231 is attached to the heat sink 270 via any of several techniques, including welding, laser welding, brazing, laser brazing, soldering, spin/friction welding, bonding, or other techniques that provide a good thermal connection between these components. One such technique includes providing an interference fit between features on the heat sink 270 and corresponding features on the electrode 231. One component may be heated and the other cooled prior to assembly, so that as the components reach equilibrium, they join tightly together. In some cases, the electrode 231 can be attached to the heat sink 231 with a thermally, conductive adhesive, in which case, the electrode 231 can include glue grooves 601. The electrode 231 can also include a tab 602 to which an electrical lead (not shown) is attached. In another embodiment, the electrode 231 and the heat sink 270 can be formed as a single unit, e.g., via a casting and/or machining process.

In other embodiments, the working portion 228 can have other arrangements. For example, the heat sink 270 can be shorter, so that the joint between the heat sink 270 and the braided shaft 603 is located within the inflatable member 260. In still another embodiment, the heat sink 270 may not be necessary, and can instead be replaced with an adapter (e.g., formed from a plastic), having a geometry generally similar to that of the heat sink 270. Accordingly, the electrode 231 can be adhesively attached to the adapter using a suitable adhesive that is carried in the glue grooves 601. In yet another embodiment, the inflatable member can be eliminated from the working portion 228. For example, in some instances (e.g., when the patient has a relatively long PFO tunnel), the electrode 231 can be inserted well within the tunnel and the vacuum drawn through the electrode 231 itself can be sufficient to form a temporary seal between the electrode 231 and the adjacent cardiac tissue during the tissue bonding or welding operation, without the need for the additional sealing action provided by the inflatable member.

C. Systems and Methods for Controlling the Application of Energy to Cardiac Tissue

FIGS. 7A-11C illustrate systems and methods for controlling the manner in which procedures are carried out on cardiac tissue, for example, the manner in which RF energy and vacuum are applied to septal tissue during a PFO closure procedure. FIG. 7A illustrates an embodiment of the control unit 240 (shown schematically in FIG. 2), which includes a console 780 and a foot unit 785. Both the console 780 and the foot unit 785 can include input devices 781 for controlling the overall system. The console 780, the foot unit 785 and the operation of the input devices 781 are described in greater detail below.

The console 780 can include a housing 782 that is clamped to a pole (not shown in FIG. 7A) to reduce the footprint occupied by the console 780, and to facilitate placement and storage of the console 780. The housing 782 carries some of the input devices 781, along with associated electronics and ports for providing services to the catheter 220, the proximal portion of which is shown in FIG. 7A. For example, the housing 782 can carry a main power switch 784 located at a rearwardly facing surface of the console 780. Positioning the main power switch 784 at the rear of the console 780 can reduce the likelihood for a practitioner to inadvertently deliver multiple doses of energy to the patient because the practitioner must take the step of reaching behind the console 780 to reset the main power switch 874 before administering a subsequent dose of energy. In other embodiments, other techniques may be employed to achieve this purpose, and in at least some of those embodiments, an alternate main power switch 784a can be positioned at the forwardly facing surface of the console 780. In yet another arrangement, the practitioner can use a separate reset switch 784b instead of the main power switch 784, 784a. In any of these embodiments, the status of the various functions provided by the console 780 can be presented at a display 783, which is described in further detail with reference to FIG. 10.

The console 780 can include a catheter power port 788 which is coupled to the catheter 220 with an electrical lead to provide power to the electrode 231 (FIG. 2). A ground pad port 788a can be coupled to a patient ground pad to complete the monopolar electrical circuit. The console 780 can also include a vacuum source port 793, which is coupled to either an external source of vacuum (e.g., a hospital-wide vacuum network, or a dedicated vacuum pump) or an internal source. For example, the console 780 can have an internal vacuum source (e.g., a vacuum pump) accessible via an internal source port 772. When the console 780 includes the internal vacuum source, the vacuum source port 793 can be connected to the internal source port 772 by simply bending the associated conduit (which terminates at the vacuum source port 793) around to attach to the internal source port 772. In any of these arrangements, the vacuum source can be configured to provide evacuation to an absolute pressure of from about 50 mm. Hg to about 300 mm. Hg, and, in a particular embodiment, about 50 mm. Hg.

The console 780 also includes a catheter vacuum port 795, which is coupled to the catheter 220 to provide the vacuum to the working portion of the catheter. A disposable collection unit 790 can be releasably attached to the console 780 to collect fluids drawn from the patient's body, thereby preventing the fluids from contaminating the vacuum source. Accordingly, the disposable collection unit 790 can include a clear-walled liquid collection vessel 791 having graduation markings 794 that indicate the volume of liquid removed from the patient during a procedure. The total volume of the liquid collection vessel 791 can be selected to be below a level of fluid that can be safely withdrawn from the patient. Accordingly, the collection vessel 791 can provide valuable information to the practitioner about the total volume of liquids withdrawn during each procedure. Such information can also include the rate at which liquids are withdrawn from the patient, which the practitioner can gauge by observing the rate at which liquids accumulate in the collection vessel 791, and/or by observing liquids passing through clear conduits of the system. In certain embodiments, the disposable collection unit 790 can also include a paddle wheel or other device that indicates the liquid flow rate to the practitioner. In any of these embodiments, the liquid collection vessel 791 can be coupled to an interface unit 792 that releasably couples the collection unit 790 to the housing 782.

In a particular embodiment, the entire collection unit 790 (e.g., both the collection vessel 791 and the interface unit 792) can be securely attached to each other to form a unitary structure so as to prevent either unit from being separated from the other, without irreparably damaging the entire collection unit 790. In another embodiment, the collection vessel 791 and the interface unit 792 can be separable from each other. An advantage of having the collection vessel 791 and the interface unit 792 inseparable from each other is that bodily fluids are less likely to leak from the collection unit, thereby reducing the likelihood for practitioners or others to come into contact with the fluids. The unitary structure is also easy for the practitioner to install and remove. Because the entire collection unit 790 is disposable (in at least one embodiment), it can also be simple and efficient for the practitioner to dispose of.

In operation, the catheter 220 is connected to the appropriate ports of the console 780, and introduced into the patient's body. The console 780 is activated by turning on the main power switch 784. Vacuum is applied to the patient by activating a vacuum switch 786 located at the foot unit 785. After an appropriate seal is achieved between the working portion of the catheter 220 and the adjacent tissue, RF energy is provided to the patient by activating an RF switch 787. The vacuum switch 786 and the RF switch 787 can be located on opposite sides of the foot unit 785 to provide the practitioner with a clear indication of which switch is which. In addition, these switches can be configured to provide other sensory cues that distinguish the switches from each other. For example, the RF switch 787 can require a higher input force for activation than does the vacuum switch 786. In a particular embodiment, the RF switch 787 may take up to ten pounds of force to activate, while the vacuum switch 786 may take less than one pound to activate.

The system can optionally include still further features to prevent the RF energy from being applied inadvertently. For example, the system can include an RF arming switch 787a that must be activated prior to activating the RF switch 787. In another arrangement, the RF switch 787 must be activated twice (once to arm and once to deliver power) before electrical energy is actually provided to the patient. In other embodiments, the vacuum switch 786, the RF switch 787, and/or other input devices of the control unit 240 can have other configurations.

The system can include other safety features in addition to or in lieu of those described above. For example, the practitioner may wish to use a different catheter and/or electrode (e.g., a smaller electrode) when performing a procedure on children than when performing the procedure on adults. A pediatric catheter can have a preselected impedance or other characteristic value that is deliberately chosen to be different than the corresponding characteristic value of an adult catheter. When the practitioner attaches the catheter to the catheter power port 788, the control unit 240 can automatically detect the nature of the catheter, and can automatically adjust certain parameters. For example, as will be described in greater detail below with reference to FIG. 10, the system can automatically set energy and/or vacuum levels. If these levels should be adjusted (e.g., made lower) for pediatric or other special applications, the system can automatically make the adjustments.

In any of the foregoing embodiments, after the procedure has been completed, the disposable collection unit 790 can be removed from the console 780 and replaced with a new disposable collection unit 790 prior to initiating a similar procedure on another patient. FIG. 7B illustrates the disposable collection unit 790 in the process of being removed from the console 780. In a particular aspect of this embodiment, the disposable collection unit 790 can be removed by simply pressing a release latch 759, rotating the collection unit, and lifting it forwardly and upwardly away from the console 780, without the use of tools.

FIG. 7C illustrates the console 780 after the disposable collection unit 790 has been removed. The console 780 can include a valve unit 750 having at least one actuator 751 that acts on the disposable collection unit 790. For example, the actuator 751 can include one or more linear actuators, rotary actuators or other suitable devices. In an embodiment shown in FIG. 7C, the valve unit 751 can include a first piston 752a and a second piston 752b, each of which operates on the disposable collection unit 790 to control the pressure in the vacuum lumen of the catheter 220 (FIG. 7A). The console 780 generally (e.g., the valve unit 750 in particular) can also include a first receiving portion 789 (e.g., a recess) that removably receives a corresponding portion of the disposable collection unit 790. The first receiving portion 789 can also include first registration features 779 that locate the disposable collection unit 790 and, in at least one embodiment, provide a simple hinge line about which the disposable collection unit 790 can be rotated. Further details of this arrangement are described below with reference to FIGS. 8A-9B.

FIG. 8A is a rear view of the disposable collection unit 790 shown in FIG. 7A, after it has been removed from the console 780 (FIG. 7C). The interface unit 792 can include a second receiving portion 896 having second registration features 897 that cooperate with the first registration features 779 shown in FIG. 7C. For example, the second registration features 897 can include closed-end channels that slip over the peg-shaped first registration features 779. Accordingly, the first and second registration features, 779, 897 may have only one engaged configuration, a configuration that is easily and readily implemented by the practitioner. The interface unit 792 can also include an interface housing 898 having multiple piston access openings 899 through which the pistons 752a, 75b (FIG. 7C) move to access corresponding fluid conduits.

FIG. 8B illustrates the valve unit 750 from the console 780 (FIG. 7C), along with the disposable collection unit 790, from which the interface housing 898 (FIG. 8A) has been removed. The interface unit 792 includes a first conduit 855a that extends between the catheter vacuum port 795 and the liquid collection vessel 791. The first conduit 855a can include a flexible material that passes adjacent to a first valve pinch point 754a. When the first piston 752a presses against the first conduit 855a at the first valve pinch point 754a, the first conduit 855a closes. Accordingly, the first piston 752a can form part of a first valve 853a. The interface unit 792 can also include a second conduit 855b connected between the first conduit 855a and an air intake or vent port 856. The second conduit 855b can pass adjacent to a second valve pinch point 754b, and can accordingly be closed when the second piston 752b is activated (the second piston 752b forming part of a second valve 853b). The interface unit 792 can still further include a third conduit 855c that extends between the liquid collection vessel 791 and the vacuum source port 793. A filter (e.g., a Gortex® filter) and/or desiccant housing 857 can be coupled between the third conduit 855c and the liquid collection vessel 791 to remove impurities and/or vapor upstream of the vacuum source, which is not shown in FIG. 8B. A filter and/or desiccant can also be provided at the air intake or vent part 856 to restrict/prevent liquid from passing into or out of the vent port 856. This arrangement can accordingly protect the vacuum source. Because the housing 857 and the vent port 856 are parts of the disposable collection unit 790, the components contained in them (e.g., the filter and/or desiccant) can be configured for a single use, and need not be maintained by the practitioner or other personnel. As a result, the apparatus can be simpler and less expensive to own and maintain than are existing devices.

In operation, both the first valve 853a and the second valve 853b are normally closed when unpowered, with the first piston 752a pinching the first conduit 855a closed at the first valve pinch point 754a, and the second piston 752b pinching the second conduit 855b closed at the second valve pinch point 754b. When the practitioner directs vacuum to be applied to the patient, the first valve 853a opens, coupling the catheter vacuum port 795 to the vacuum source port 793. At this point, vacuum is drawn through the catheter vacuum port 795, the first conduit 855a, the liquid collection vessel 791, the third conduit 855c and the vacuum source port 793, as indicated by arrows in FIG. 8B, to clamp the patient's cardiac tissue against the electrode 231 (FIG. 5B). After the PFO sealing procedure has been completed, the first valve 853a closes, cutting off communication between the vacuum source and the catheter 220 (FIG. 5B). However, the pressure at the catheter vacuum port 795 and in the catheter 220 itself will typically remain below atmospheric pressure. Accordingly, the second valve 85b can open to vent the catheter vacuum port 785 and the catheter 220 to atmospheric pressure, via the second conduit 85b and the air intake port 856. When the catheter is open to atmospheric pressure, the vacuum seal between the cardiac tissue and the electrode 231 is released, allowing the practitioner to remove or reposition the electrode 231. After a suitable venting period, the second valve 85b can automatically return to its closed state. This arrangement can save power (e.g., when the second valve 85b is a normally closed valve that is unpowered when closed) and can prevent fluids from escaping from the patient's body through the catheter 220.

One feature of an embodiment of the disposable collection unit 790 is that it includes the conduits 85an, 85b. Another feature is that the conduits 85a, 85b have fixed positions that are consistent from one unit 790 to the next. The corresponding valves 85a, 85b (in the console 780) also have fixed positions. Another feature is that the conduits 85a, 85b are configured for a single use. The foregoing features differ from existing pinch valve arrangements, in which a practitioner typically stretches and installs a length of flexible tubing into the pinch valve, and may use the tubing over and over. A drawback with the existing pinch valve arrangement is that if the practitioner fails to install the flexible tubing properly or consistently (an event which is made more likely because the tubing must be stretched), the valves will not operate properly. An advantage of an embodiment of the invention described above is that the conduits 85a, 85b are installed at the time of manufacture, are disposable, and need not be manipulated by the practitioner during use.

Another feature of the disposable collection unit 790 and the console 780 is that the patient's bodily fluids are contained by and come in contact with only the disposable single-use collection unit 790 and not the rest of the multi-use console 780. An advantage of this arrangement is that it is easy for the practitioner to use, and it reduces if not eliminates the likelihood for contacting the practitioner (or a subsequent patient) with the bodily fluids of the patient currently undergoing the procedure.

FIGS. 9A and 9B schematically illustrate the first and second valves 85a, 85b, along with an activation diagram that depicts operation of the valves in accordance with an embodiment of the invention. When a “VAC ON” input signal is received (e.g., when the practitioner activates the vacuum switch 786 shown in FIG. 7A), the first valve 85a opens to allow communication between the vacuum source port 793 and the catheter vacuum port 795. When a “VAC OFF” input signal is received (e.g., when the practitioner re-activates the vacuum switch 786), the first valve 85a closes, and the second valve 85b opens to vent the catheter 220. In a particular embodiment, the second valve 85b can remain open for a period of from about two to about five seconds to allow full venting of the catheter, after which the second valve 85b automatically closes. Both the first valve 85a and the second valve 85b can then remain closed until a new “VAC ON” input is received.

One feature of an embodiment of the arrangement described above is that the system can automatically vent the catheter to atmospheric pressure upon receiving a signal to deactivate the application of vacuum to the patient. For example, the system can include one or more computer-readable media containing instructions that direct the automatic operation of the valves. This automated feature can have several advantages. For example, this feature can allow the practitioner to quickly and automatically vent the catheter to (or at least toward) atmospheric pressure, which in turn allows the practitioner to quickly move the electrode within the body (if necessary), or remove the catheter from the patient's body after completing a procedure. Because the operation is automatic, it can reduce or eliminate the likelihood that the practitioner will attempt to move the electrode while vacuum is still applied. This feature can therefore reduce the likelihood for damage to the patient's cardiac tissue.

Another feature of an embodiment of the foregoing arrangement is that the automatic operation of the valves can be quicker than conventional manual techniques. An advantage of this feature is that it can reduce patient blood loss during a procedure. Another advantage is that it can reduce the amount of time required to reposition the catheter (if necessary), and therefore reduce the time required to complete the procedure.

Another feature of an embodiment described above is that the second valve 85b can automatically open at the same time the first 85a valve is closing. An advantage of this feature is that it can reduce the likelihood for the catheter and/or cable/tubing assembly to “buck” or move suddenly when the vacuum is suddenly removed. As a result, the practitioner can maintain control of the catheter without having to manually open one valve while simultaneously and manually closing the other.

Certain aspects of the embodiments described above with reference to FIGS. 7A-9B include a vacuum source that provides a generally continuous, generally constant level of vacuum to the catheter. In other embodiments, the vacuum can be applied in other manners. For example, instead of a vacuum pump, the collection vessel 791 shown in FIG. 7A can be pre-evacuated prior to use, and can have a volume sufficient to provide vacuum over the course of an entire procedure (e.g., from 1-9 minutes, 1-5 minutes, or up to about 2 minutes for a single procedure). In a particular application, the collection vessel 791 has a volume of from about one-half pint to about three pints (e.g., about one pint or less). The volume of the collection vessel 791 may not need to be larger because once a firm seal is established between the catheter and the patient's tissue, the pressure in the vessel 791 should remain approximately constant. The absolute pressure in the vessel 791 can be from about 50 mm. Hg to about 300 mm. Hg, and in a particular embodiment, about 50 mm. Hg. The other portions of the disposable collection unit 790 and the console 780 can be generally similar to those described above, except that the third conduit 85c (FIG. 8B) and the vacuum source port 793 (FIG. 8B) can be eliminated. In use, the pressure within the collection vessel 791 will only increase or remain constant over at least some time intervals. In fact, an advantage of the pre-evacuated, single use collection vessel 791 is that it can eliminate the need for an on-site vacuum pump or other high-volume vacuum source.

FIG. 10 is a partially schematic illustration of the information presented to the practitioner at the display 783 of the console 780 during a representative procedure, independent of the manner in which vacuum is provided to the catheter. The display 783 can present a remaining treatment time indicator 1078 (indicating the amount of time remaining during which the electrode or other energy transmitter is active). A representative treatment time for a PFO sealing procedure is 2 minutes, though treatment times can be less, or (as described above) can range up to or beyond 9 minutes in some cases. Different treatment times may be appropriate for procedures other than PFO sealing procedures. In any of these cases, if the treatment is halted prior to normal completion, the remaining treatment time indicator 1078 can remain visible for a predetermined time to allow the practitioner to record the indicated value. Alternatively, the indicated value can remain visible until the practitioner resets the system via the main power switch 784 or the reset switch 78b. An “RF On” indicator 1074 indicates when the electrode is active, and a “Vac On” indicator 1077 indicates when vacuum is active. A “Treatment End” indicator 1075 identifies when the treatment is over, and a “Low Vacuum” indicator 1076 indicates when the vacuum is outside a target range (e.g., if there is a leak in the system that prevents sufficient vacuum from being drawn on the patient). For example, if the absolute pressure exceeds a target value in the range of from about 250 mm Hg to about 300 mm Hg, as measured by an appropriately positioned pressure transducer, the “Low Vacuum” indicator 10b can illuminate or otherwise activate. The system can automatically prevent the corresponding valve (e.g., the first valve 85a, shown in FIG. 9) from opening until a sufficient vacuum level is restored. Optionally, the console 780 can also include an “RF armed” indicator 1073 for example, if the operator must first arm the RF delivery function before activating it. In such a case, the foot unit 785 (FIG. 7A) can include the RF arming switch 78a. The RF armed indicator 1073 can be visible (as shown in FIG. 10) and/or audible. As shown in FIG. 10, the information displayed to the practitioner and the available options for the practitioner can be relatively simple and straightforward. Further details of embodiments that include these features are described below with reference to FIGS. 11A-11C.

FIG. 11A is a schematic block diagram of a system 1100 for applying treatment to a patient in accordance with an embodiment of the invention. The system 1100 can include a power delivery component 1101 (e.g., an RF generator and associated circuitry) that directs energy to the patient. The power delivery component 1101 can be activated by an activation device 1102, which in turn responds to a user input 1105. For example, the activation device 1102 can include the RF switch 787 described above with reference to FIG. 7A. In a particular aspect of an embodiment shown in FIG. 11A, the amount of energy supplied to the patient once the user activates the activation device 1102 can be fixed (e.g., at the time of manufacture) so as not to be changed by the practitioner, patient, or any other user. The amount of energy (the product of current, voltage and delivery time) can correspond to the amount typically required to seal a PFO or conduct another pre-defined cardiac tissue procedure. For a system that delivers energy at a constant current and voltage, the energy dose is determined solely by the length of time the energy is being delivered. In other systems, for which voltage and/or current vary, the treatment time may also vary, so the system may be configured to calculate a running total of energy delivered, and halt the delivery when the pre-defined energy dose is reached. A typical range of energies for a single dose is from about 10 joules to about 6500 joules. For example, in one embodiment 12 watts of power is provided for a period of two minutes, for a total energy dose of 1440 joules. In any of these arrangements, by automatically terminating the delivery of energy to the patient after the fixed amount has been delivered, the system 1100 can predictably and repeatedly deliver fixed doses of energy to a series of patients, thereby improving the reliability of the results achieved by the procedure. This can also be simpler for the practitioner to operate, because the practitioner need not calculate and input parameters such as signal voltage and/or treatment time, as is common with existing devices.

Parameters in addition to or in lieu of the total applied energy can also be automatically established and set, further reducing the workload on the practitioner. For example, the system 1100 can automatically set the level of vacuum applied to the catheter. In a particular embodiment, the absolute pressure can be from about 50 mm Hg to about 300 mm Hg at the patient's tissue, independent of the local atmospheric pressure. This level is expected to provide suitable clamping between the catheter and the adjacent tissue, without causing undue foaming in the liquids removed from the patient's body. In other embodiments, the vacuum level can be different and/or the system 1100 can automatically set other parameters.

Of course, the system 1100 can include facilities for overriding the automatic delivery of energy to the patient. For example, the system 1100 can include a manual interrupt device 1103 that responds to a user interruption input 1106. In a particular embodiment, the user (e.g., the practitioner) can interrupt the energy provided to the patient by resetting the power switch 784 (FIG. 7A), the reset switch 78b (FIG. 7A), or the RF switch 787 (FIG. 7A). Accordingly, the practitioner can quickly halt the delivery of energy in response to some indication that such an action is warranted. In another embodiment, the system 1100 can include an automatic interrupt device 1104 that responds to a sensor input 1107. For example, the sensor input 1107 can provide an indication of an open circuit, a short circuit, an impedance rise, a high temperature, a loss of vacuum, or another occurrence in light of which it is advisable to cease delivering energy to the patient.

The operation of the vacuum can be automatically tied to the application of energy to the patient, in particular embodiments. For example, in one arrangement, the system can include an electronic (or other) lockout that automatically prevents the vacuum from being turned off for a predetermined time interval following the end of energy delivery to the patient. In a particular aspect of this arrangement, the time interval is about 5 seconds, but the time interval can have other (shorter or longer) intervals as well. An advantage of this arrangement is that it precludes the practitioner from removing the energy delivery device from the patient until the energy delivery device has had an opportunity to cool down by a selected amount.

FIG. 11B is a flow diagram illustrating an embodiment of a process 1120 for treating a patient, and includes reference to particular elements and functions of the systems and devices described above. Process portion 1121 can include receiving a request to initiate vacuum, e.g., via the vacuum switch 786 (FIG. 7A). In response to the request, process portion 1122 includes directing the initiation of vacuum. In process portion 1123, the vacuum is monitored and the results are displayed. For example, the results can be displayed by illuminating the “Vacuum On” indicator 1077 shown in FIG. 10, and/or the “Low Vacuum” indicator 1076. In process portion 1124, a request is received to initiate the delivery of energy, in response to which energy delivery is initiated. In process portion 1125, the system can check to determine whether an interrupt request has been received. The interrupt request can either be automatically generated or manually generated. In either instance, if an interrupt request is received, the treatment procedure is automatically terminated (process portion 1129). If not, process portion 1126 includes determining the delivered dose and displaying some representation of the delivered dose to the practitioner. This display can include an amount of time elapsed, an amount of energy applied, or, as shown in FIG. 10, an amount of time remaining until a complete dose has been delivered. In process portion 1127, the delivered dose is compared to a pre-set dose. If the delivered dose meets or exceeds the pre-set dose (process portion 1128), the procedure is automatically terminated (process portion 1129). Otherwise, the process returns to process portion 1126.

Once the process has been automatically terminated (process portion 1129), the system can check to see if a reset request has been received (process portion 1130). A reset request can include shutting the system off by tripping the main power supply switch 784 (FIG. 7A), or by activating another reset device. If such a request is received, the dose is reset (process portion 1131) and the procedure returns to process portion 1121.

In several embodiments described above, the effect of the cardiac tissue undergoing an increase in impedance (e.g., “impeding out”) is an effect to be avoided because it may prevent RF energy from subsequently penetrating into the adjacent tissue. In other embodiments, for example, when heat is transferred efficiently and effectively away from the electrode, an impedance increase may be used to indicate the completion of a suitable energy dose. FIG. 11C illustrates a process in accordance with one such embodiment. The process can include receiving a request to initiate the delivery of energy (process portion 1124) and in response, delivering an initial energy dose (process portion 1140). The impedance of the electrical circuit that includes the treated tissue can be monitored on a continuous or intermittent basis, or detected after the initial energy dose has been delivered (process portion 1141). The impedance can be measured by any suitable technique, including determining a change in the voltage drop across the treated tissue. In process portion 1141, it is determined whether the impedance has achieved a target value and/or has changed by a target amount. For example, process portion 1142 can include determining whether the impedance has increased to a predetermined threshold level, and/or determining whether the impedance has changed by a threshold amount. If the impedance has changed by or to the target value, the treatment is effectively complete and the process can further include resetting the dose in preparation for treating a subsequent patient (process portion 1144). If not, process portion 1143 can include delivering a follow-on energy dose. Process portions 1141-1143 can be repeated until the impedance value corresponds to a value indicating a completed treatment. Although not shown in FIG. 11C, other features described above with reference to 11B (e.g., determining whether an interrupt request has been received and displaying results) can be included in embodiments of the method shown in FIG. 11C.

FIG. 12 is a side elevation view of a liquid collection vessel 1291 that includes features in accordance with further embodiments of the invention. The liquid collection vessel 1291 can be compatible with other features of the disposable collection unit 790 described above. Accordingly, the vessel 1291 can include a first conduit 125a that can be coupled to the vacuum channel of the catheter, and a third conduit 125c that can be coupled to the vacuum source. The first conduit 125a can extend through the vessel 1291 toward the bottom of the vessel 1291. A core 1249 (e.g., a porous core formed from a polymer) can be positioned between the open end of the first conduit 125a and the open end of the third conduit 125c. The core 1412 can be supported in position by one or more retention rings 1247 (two are shown in FIG. 12). When blood is withdrawn from the patient through the catheter, it is directed by the first conduit 125a to the bottom of the vessel 1291. As the result of the vacuum drawn on the third conduit 125c, the blood may tend to foam or bubble up. By positioning the core 1249 between the bottom of the vessel 1291 and the opening of the third conduit 125c, the likelihood for the foam to enter the third conduit 125c and contaminate the vacuum source can be reduced or eliminated.

In a further aspect of an embodiment shown in FIG. 12, the core 1249 can be impregnated with an antifoaming agent or a surfactant, for example, an agent that includes silicone oil. In a further aspect of this embodiment, the antifoaming agent can be initially contained in a rupturable capsule 1248 placed in the vessel 1291 between the bottom of the vessel and the core 1249 at the time of manufacture. Accordingly, the antifoaming agent can be contained in the capsule 1248 until just prior to use. The capsule 1248 can burst under the influence of the vacuum drawn through the third conduit 125c, releasing the antifoaming agent into the vessel 1291, where it can coat the core 1249 and further reduce the likelihood for foam to contaminate the vacuum source. In other embodiments, the antifoaming agent can be housed in other portions of the overall system. For example, the antifoaming agent can be housed in the interface unit 792 (FIG. 7A), or injected through the interface unit 792 through the vacuum port 795 (FIG. 7A), prior to applying vacuum to the disposable collection unit 790 (FIG. 7A).

In any of the foregoing embodiments, including that shown in FIG. 12, the level of vacuum applied to the catheter can also be selected to produce suitable performance while controlling the amount of liquid foaming. In a particular embodiment, the absolute pressure can be selected to be within the range of about 50 mm Hg to about 150 mm Hg (absolute). In a further particular embodiment, the absolute pressure can have a value of no less than about 50 mm Hg to avoid foaming and/or boiling. These levels can be adjusted as needed, for example, to account for different altitudes.

From the foregoing, it will be appreciated that specific embodiments of the invention have been described herein for purposes of illustration, but that various modifications may be made without deviating from the invention. For example, the electrodes, inflatable members, disposable collection units, and/or other components of the overall systems described above can have other shapes, sizes, and/or configurations in other embodiments. In particular embodiments, the inflatable members, energy transmitters and/or guidewire conduits described above are arranged asymmetrically with respect to the terminal axis, while in other embodiments, some or all of these components can be symmetric with respect to the terminal axis (e.g., the inflatable member can have a round shape that is concentric with the terminal axis). The energy transmitter can be configured to deliver bipolar rather than monopolar signals, for example, via multiple electrodes positioned at or near the PFO. Furthermore, while the devices described above were described principally in the context of a PFO repair procedure, devices and techniques generally similar to those described above may be used in other treatment contexts. For example, some or all aspects of the console and the valve arrangements described in the context of a PFO repair procedure with respect to FIGS. 7A-11 may be applied in other contexts (cardiovascular or otherwise) in other embodiments. Aspects of the invention described in the context of particular embodiments may be combined or eliminated in other embodiments. Further, while advantages associated with certain embodiments of the invention have been described in the context of those embodiments, other embodiments may also exhibit such advantages, and not all embodiments need necessarily exhibit such advantages to fall within the scope of the invention. Accordingly, the invention is not limited except as by the appended claims.

I/We claims:

1. A patient treatment device, comprising:
a catheter having a proximal end and a distal end, the catheter including a working portion positioned toward the distal end and being elongated along a terminal axis; and
an energy transmitter at the working portion of the catheter, the energy transmitter being tapered inwardly toward the terminal axis in a distal direction, the energy transmitter being asymmetric relative to the terminal axis.

2. The device of claim 1 wherein the energy transmitter includes an electrode.

3. The device of claim 1 wherein the energy transmitter has an asymmetrical conical shape.

4. The device of claim 1 wherein an external surface of the energy transmitter has an asymmetrical conical shape disposed outwardly from the terminal axis, and wherein a first part of the external surface is generally parallel to the terminal axis, and a second part of the external surface is non-parallel to the terminal axis.

5. The device of claim 4 wherein the first part of the external surface has a concave recess.

6. The device of claim 1 wherein the energy transmitter includes at least one vacuum aperture coupleable to a vacuum source.

7. The device of claim 1 wherein the energy transmitter includes a plurality of vacuum apertures coupleable to a vacuum source, and wherein the vacuum apertures have a slot shape aligned generally parallel with the terminal axis.

8. The device of claim 1 wherein the energy transmitter has an internal guidewire conduit positioned to slideably receive a guidewire, and wherein the guidewire conduit is non-parallel to the terminal axis.

9. The device of claim 1, further comprising an inflatable member proximate to the energy transmitter, the inflatable member being asymmetric relative to the terminal axis.

10. The device of claim 1 wherein the working portion has a non-zero bend angle relative to a portion of the catheter immediately adjacent in a proximal direction.

11. The device of claim 1 wherein the working portion has a non-zero bend angle relative to a portion of the catheter immediately adjacent in a proximal direction, and wherein the energy transmitter is symmetric relative to a plane that includes the non-zero bend angle and the terminal axis.

12. The device of claim 1, further comprising:
a heat sink carried by the working portion of the catheter, the heat sink being in thermal communication with the energy transmitter; and
an electrically resistive, thermally conductive material disposed around an outer surface of the heat sink.

13. The device of claim 12 wherein the heat sink has a generally hollow cylindrical shape.

14. The device of claim 12 wherein the energy transmitter includes an electrode, and wherein the heat sink and the electrode are integral with each other.

15. The device of claim 14 wherein the heat sink is at least partially electrically isolated from the electrode.

16. The device of claim 14, further comprising an electrically resistive, thermally conductive material positioned between the electrode and the heat sink.

17. The device of claim 12, further comprising an inflatable member carried by the working portion, the inflatable member being coupleable to a source of pressurized liquid, and wherein the heat sink is in thermal communication with the inflatable member.

18. The device of claim 17 wherein the heat sink is in direct thermal communication with an interior region of the inflatable member.

19. The device of claim 17 wherein a first portion of the heat sink is in thermal communication with the inflatable member and is in direct contact with the inflatable member, and wherein a second portion of the heat sink is exposed and out of direct contact with the inflatable member.

20. The device of claim 17 wherein at least one of the energy transmitter and the inflatable member is asymmetric relative to the terminal axis.

21. The device of claim 17 wherein both the energy transmitter and the inflatable member are asymmetric relative to the terminal axis, and wherein the energy transmitter and the inflatable member are symmetric about a common plane of symmetry.

22. The device of claim 12, further comprising an inflatable member proximate to the energy transmitter and disposed circumferentially around the working portion of the catheter, and wherein the heat sink has a generally hollow cylindrical shape, further wherein the heat sink is positioned in an annular region adjacent to an inner surface of the inflatable member.

23. The device of claim 12 wherein the heat sink includes at least one of silver and a silver alloy.

24. The device of claim 12 wherein the heat sink includes a gold-plated copper-silver alloy.

25. The device of claim 12 wherein the heat sink is configured to transfer sufficient heat away form the energy transmitter to keep a temperature increase of the energy transmitter to 10° C. or less, per watt of energy removed by the heat sink.

26. A patient treatment device, comprising:
a catheter having a proximal end and a distal end, the catheter including a working portion positioned toward the distal end and being elongated along a terminal axis;
an energy transmitter at the working portion of the catheter; and
a guidewire conduit carried by the working portion of the catheter, the guidewire conduit being positioned to receive a guidewire and being non-parallel to the terminal axis.

27. The device of claim 26 wherein the energy transmitter includes an electrode.

28. The device of claim 26 wherein the guidewire conduit extends through the energy transmitter.

29. The device of claim 26 wherein the guidewire conduit is oriented at an angle of from about 3° to about 20° relative to the terminal axis.

30. The device of claim 26 wherein the guidewire conduit is oriented at an angle of about 9° relative to the terminal axis.

31. The device of claim 26 wherein the energy transmitter has an asymmetric tapered shape relative to the terminal axis, and wherein the energy transmitter is symmetric about a plane that includes the terminal axis and the portion of the guidewire conduit that is non-parallel to the terminal axis.

32. The device of claim 26, further comprising an inflatable member proximate to the energy transmitter, the inflatable member being asymmetric relative to the terminal axis.

33. A device for treating a patent foramen ovale, comprising:
a catheter having a proximal end and a distal end, the catheter including a working portion positioned toward the distal end and being elongated along a terminal axis, the working portion having a non-zero bend angle relative to the immediately adjacent portion of the catheter;
an electrode at the working portion of the catheter, the electrode including multiple, slot-shaped vacuum ports, the electrode being asymmetric relative to the terminal axis, wherein a first surface of the electrode is oriented at a first acute angle relative to the terminal axis, and an oppositely-facing second surface of the electrode is oriented approximately parallel to the terminal axis;
a guidewire conduit positioned within the electrode, the guidewire conduit being oriented at a second acute angle relative to the terminal axis; and
an inflatable member proximate to the electrode, the inflatable member being asymmetric relative to the terminal axis and having a first portion with an outer surface extending from the terminal axis by a first distance and a second, oppositely-facing portion with an outer surface extending from the terminal axis by a second distance greater than the first distance; and wherein
the bend angle, the first acute angle, the second acute angle, the first distance and the second distance are located at least approximately in the same plane.

34. The device of claim 33 wherein the first acute angle has a value of from 20° to about 50°

35. The device of daim 33 wherein the second acute angle has a value of bout 3° to about 20°

36. The device of claim 33, further comprising a heat sink carried by the al portion, the heat sink being in thermal communication with both the electrode and flatable member, the heat sink being at least partially electrically isolated from the elctrode.

37. A method for treating a patent foramen ovale located between a septum primum and a septum secundum, comprising:
positioning a working portion of a catheter at least proximate to the patent foramen ovale, the working portion being elongated along a terminal axis;
orienting an energy transmitter carried by the working portion relative to the patent foramen ovale so that a first tapered surface of the energy transmitter has a different angular orientation relative to the terminal axis than does a second tapered surface of the energy transmitter; and contacting the first tapered surface of the energy transmitter with the septum primum and contacting the second tapered surface of the energy transmitter with the septum secundum; and
activating the energy transmitter to at least partially seal the patent foramen ovale.

38. The method of claim 37 wherein orienting an energy transmitter includes orienting an electrode.

39. The method of claim 37, further comprising drawing a vacuum on at least one of the septum secundum and the septum primum.

40. The method of claim 37, further comprising drawing the septum secundum and the septum primum into contact with the energy transmitter by drawing a vacuum through apertures in an external surface of the energy transmitter.

41. The method of claim 37, further comprising:
inflating an inflatable member located proximate to the energy transmitter; and
sealably engaging the inflatable member with the tissue adjacent to the patent foramen ovale.

42. The method of claim 41, further comprising moving the energy transmitter relative to the inflatable member along the terminal axis.

43. The method of claim 41 wherein the patent foramen ovale includes a PFO tunnel, and wherein the method further comprises positioning the inflatable member so that it contacts cardiac tissue external to the PFO tunnel while the energy transmitter extends at least partially into the PFO tunnel.

44. The method of claim 37, further comprising guiding the energy transmitter into contact with the tissue adjacent to the patent foramen ovale by sliding the energy transmitter along a guidewire that passes through the energy transmitter.

45. The method of claim 44 wherein sliding the energy transmitter includes sliding the energy transmitter into contact with the tissue adjacent to the patent foramen ovale along an axis that is oriented at a non-zero angle relative to the terminal axis.

46. The method of claim 45, further comprising preferentially urging the guidewire into contact with the secundum and away from the primum.

47. The method of claim 37, further comprising moving the energy transmitter toward the patent foramen ovale while the energy transmitter is fixed spatially relative to a bend in the catheter located proximate to the energy transmitter.

48. The method of claim 37, further comprising contacting the septum with a saddle-shaped portion of the energy transmitter.

49. The method of claim 37 wherein orienting the energy transmitter includes orienting the second portion of the energy transmitter to be more closely aligned with the terminal axis than is the first portion of the energy transmitter.

50. The method of claim 37, further comprising:
guiding the catheter toward the patent foramen ovale by sliding the energy transmitter along a guidewire that passes through the energy transmitter at a non-zero angle relative to the terminal axis,
inflating an inflatable member located proximate to the energy transmitter, the inflatable member being asymmetric relative to the terminal axis; and
sealably contacting the inflatable member with the tissue adjacent to the patent foramen ovale while the inflatable member and the energy transmitter are fixed spatially relative to each other, and while both are symmetric about a common plane of symmetry.

51. A method for treating a patent foramen ovale located between a septum primum and a septum secundum, the method comprising:
positioning a working portion of a catheter at least proximate to the patent foramen ovale;
positioning an energy transmitter carried by the working portion proximate to the septum primum and the septum secondum;
directing the energy from the energy transmitter to the septum primum and the septum secondum to at least partially seal the patent foramen ovale;
and
while at least partially sealing the patent foramen ovale, removing sufficient energy from the energy transmitter along a path away from an interface between the energy transmitter and adjacent cardiac tissue to maintain the energy transmitter at a temperature within about 6° C. of the patient's body temperature.

52. The method of claim 51 wherein removing energy from the energy transmitter includes removing the energy via a heat sink, in a proximal direction away from the energy transmitter.

53. The method of claim 51, wherein the energy transmitter includes an electrode, and wherein the method further comprises drawing a vacuum through apertures in the electrode to draw the adjacent cardiac tissue toward the electrode.

54. The method of claim 51 wherein directing energy includes directing about 10 watts of energy.

55. The method of claim 51 wherein maintaining the energy transmitter at a temperature includes maintaining the energy transmitter at a temperature within about 4° C. of the patient's body temperature.
